(19) United States
(12) Patent Application Publication
(10) Pub. No.: US 2010/0077495 A1
(43) Pub. Date: Mar. 25, 2010

(54) COMPOSITIONS AND METHODS FOR THE EXPRESSION OF NUCLEIC ACIDS

(76) Inventors: David P. Davis, San Bruno, CA (US); Daniel C. Gray, San Franciso, CA (US); Zhenyu Gu, Shanghai (CN)

Correspondence Address: GENENTECH, INC., 1 DNA WAY, SOUTH SAN FRANCISCO, CA, 94080, US

(21) Appl. No.: 12/519,349

(22) PCT Filed: Dec. 4, 2007
(86) PCT No.: PCT/US07/86417

§ 371 Date: Nov. 18, 2009

Related U.S. Application Data
(60) Provisional application No. 60/871,390, filed on Dec. 21, 2006.

(51) Int. Cl. C12N 15/89; C12N 15/63; A01K 67/027; C12N 5/10

(52) U.S. Cl. 800/14; 435/320.1; 800/25; 435/325; 435/354

(57) ABSTRACT

Compositions and methods are provided herein for the expression of nucleic acids. Compositions and methods are also provided herein for inducible expression of nucleic acids in transgenic cells and animals using transposon-based nucleic acid constructs. Compositions and methods are also provided herein for modulation of endogenous gene expression.

### RELATED APPLICATION

This application claims the benefit of U.S. Provisional Application No. 60/871,390, filed Dec. 21, 2006, the disclosure of which is incorporated herein by reference in its entirety.

### FIELD OF THE INVENTION

The present invention relates to inducible expression of nucleic acids in transgenic cells and animals using transposon-based nucleic acid constructs.

### BACKGROUND

The ability to modulate gene expression in cells and in animals is useful for the study of gene function. For example, the function of a gene may be elucidated by expressing the gene in a cell that otherwise does not substantially express the gene or by expressing the gene to a level that exceeds the expression level normally observed in the cell. Additionally, the function of a gene may be elucidated by inhibiting or “knocking down” expression of the gene in a cell that otherwise expresses that gene.

Although conventional gene targeting methods have proven useful for disrupting gene function in mammals such as mice, such methods may sometimes yield only limited information on gene function. For example, disruption of a gene by conventional gene targeting methods in mice may result in early embryonic lethality, thus yielding limited if any information on the function of the gene at later stages of mouse development and in the adult mouse. The ability to inducibly or conditionally disrupt gene function at selected developmental stages would provide considerably more information on gene function and would further identify targets for therapeutic interventions aimed at compensating for genetic deficiencies.

RNA interference (RNAi) is a method for modulating gene expression. However, the use of RNAi has been hampered by the lack of reliable methods for efficient delivery and/or inducible expression of RNA molecules, such as siRNA, to cells and/or animals. The ability to achieve efficient delivery and/or inducible expression of RNA molecules to cellular systems would render RNAi a powerful functional genomics tool for conditional knock-down of gene function. Furthermore, the ability to selectively inhibit target gene expression has important therapeutic implications, e.g., to prevent the production of proteins that are harmful to an animal.

Thus, there exists a need for reliable and efficient methods for expressing nucleic acids and modulating gene expression in cells and animals. The present invention satisfies the above-described needs and provides other benefits.

### SUMMARY

Compositions and methods are provided herein for the expression of nucleic acids. In certain embodiments, such compositions and methods allow for inducible expression of nucleic acids from transposon-based constructs.

In one aspect, a nucleic acid construct is provided, wherein the nucleic acid construct comprises (1) a polynucleotide operably linked to an inducible promoter and (2) transposon-derived inverted repeats flanking the polynucleotide. In one embodiment, the transposon-derived inverted repeats are piggyBac inverted repeats. In another embodiment, the polynucleotide encodes a regulatory RNA, e.g., an shRNA.

In another aspect, a nucleic acid construct is provided, wherein the nucleic acid construct comprises (a) a first transcription unit comprising a polynucleotide operably linked to an inducible promoter, wherein the inducible promoter comprises one or more TetO sequences; (b) a second transcription unit comprising a coding sequence encoding a TetR; and (c) a pair of inverted repeats, wherein one of the inverted repeats is 5′ of (a) and (b), and the other of the inverted repeats is 3′ of (a) and (b). In one embodiment, the pair of inverted repeats are piggyBac inverted repeats. In one such embodiment, the one or the other of the inverted repeats comprises a polynucleotide sequence selected from SEQ ID NO:1, SEQ ID NO:2, SEQ ID NO:3, SEQ ID NO:4, and SEQ ID NO:5.

In certain embodiments, the polynucleotide encodes a regulatory RNA. In one such embodiment, the regulatory RNA is an shRNA.

In certain embodiments, the inducible promoter further comprises an H1 or U6 promoter. In one such embodiment, the inducible promoter further comprises an H1 promoter. In certain embodiments, the inducible promoter comprises at least two TetO sequences. In one such embodiment, the inducible promoter comprises an H1 promoter operably linked to at least two TetO sequences. In one such embodiment, the inducible promoter comprises the nucleic acid sequence of SEQ ID NO:16.

In certain embodiments, the polynucleotide encodes a first RNA, and the nucleic acid construct further comprises a third transcription unit, wherein the third transcription unit comprises a second polynucleotide operably linked to an inducible promoter, wherein the second polynucleotide encodes a second RNA, wherein the first RNA and the second RNA comprise sequences of at least 10 contiguous nucleotides that are complementary.

In certain embodiments, the nucleic acid construct further comprises a selectable marker. In certain embodiments, the second transcription unit further comprises a selectable marker. In one such embodiment, the selectable marker confers resistance to puromycin. In another of such embodiments, an IRES is disposed between the coding sequence encoding a TetR and the selectable marker.

In certain embodiments, the coding sequence encoding a TetR is codon-optimized. In one such embodiment, the coding sequence encoding a TetR comprises the nucleic acid sequence of nucleotides 1-507 of SEQ ID NO:15.

In yet another aspect, a method of expressing a polynucleotide in a cell is provided, the method comprising (a) introducing into the cell a nucleic acid construct comprising (i) a first transcription unit comprising the polynucleotide operably linked to an inducible promoter, wherein the inducible promoter comprises one or more TetO sequences; (ii) a second transcription unit comprising a coding sequence encoding a TetR; and (iii) a pair of inverted repeats, wherein one of the inverted repeats is 5′ of (i) and (ii), and the other of the inverted repeats is 3′ of (i) and (ii); and (b) exposing the cell to an inducing agent that induces expression of the polynucleotide from the inducible promoter. In one embodiment, the pair of inverted repeats are piggyBac inverted repeats. In one such embodiment, the one or the other of the inverted repeats comprises a polynucleotide sequence selected from SEQ ID NO:1, SEQ ID NO:2, SEQ ID NO:3, SEQ ID NO:4, and SEQ ID NO:5.

In certain embodiments, the polynucleotide encodes a regulatory RNA. In one such embodiment, the regulatory RNA is an shRNA.

In certain embodiments, the method further comprises introducing into the cell a polynucleotide encoding a transposase that acts on the inverted repeats to mediate nucleic acid transposition. In one such embodiment, the transposase is a piggyBac transposase. In one such embodiment, the transposase comprises (a) an amino acid sequence having at least 90% amino acid sequence identity to SEQ ID NO:14, or (b) a fragment of (a).

In yet another aspect, a method of inhibiting expression of an endogenous gene in a cell, the method comprising: (a) introducing into the cell a nucleic acid construct comprising: (i) a first transcription unit comprising a polynucleotide operably linked to an inducible promoter, wherein the polynucleotide encodes a regulatory RNA specific for the endogenous gene; and (ii) a pair of inverted repeats, wherein one of the inverted repeats is 5′ of (i), and the other of the inverted repeats is 3′ of (i); and (b) exposing the cell to an inducing agent that induces expression of the polynucleotide from the inducible promoter. In certain embodiments, the cell is an embryonic cell.

In certain embodiments, the regulatory RNA is an shRNA. In one such embodiment, the shRNA is specific for an endogenous gene selected from (a) a gene encoding lipin, (b) a gene encoding VEGF, or (c) a gene that is an oncogene.

In certain embodiments, the inducible promoter comprises one or more TetO sequences, the nucleic acid construct further comprises a second transcription unit comprising a coding sequence encoding a TetR, and the one of the inverted repeats is 5′ of the second transcription unit, and the other of the inverted repeats is 3′ of the second transcription unit.

In certain embodiments, the inverted repeats are piggyBac inverted repeats. In one such embodiment, the one or the other of the inverted repeats comprises a polynucleotide sequence selected from SEQ ID NO:1, SEQ ID NO:2, SEQ ID NO:3, SEQ ID NO:4, and SEQ ID NO:5. In certain embodiments, the method further comprises introducing into the cell a polynucleotide encoding a piggyBac transposase. In one such embodiment, the piggyBac transposase comprises (a) an amino acid sequence having at least 90% amino acid sequence identity to SEQ ID NO:14, or (b) an active fragment of (a).

In certain embodiments, the second transcription unit further comprises a selectable marker. In one such embodiment, an IRES is disposed between the coding sequence encoding a TetR and the selectable marker. In certain embodiments, the coding sequence encoding a TetR is codon-optimized. In one such embodiment, the coding sequence encoding a TetR comprises the nucleic acid sequence of nucleotides 1-507 of SEQ ID NO:15.

In certain embodiments, the inducible promoter comprises an H1 or U6 promoter. In certain embodiments, the inducible promoter comprises at least two TetO sequences. In one such embodiment, the inducible promoter comprises the nucleic acid sequence of SEQ ID NO:16.

In yet another aspect, a method of expressing a polynucleotide in a transgenic mammal is provided, the method comprising: (a) introducing into a mammalian, non-human embryonic cell a nucleic acid construct comprising: (i) a first transcription unit comprising the polynucleotide operably linked to an inducible promoter, wherein the polynucleotide encodes a regulatory RNA specific for the endogenous gene; and (ii) a pair of inverted repeats, wherein one of the inverted repeats is 5′ of (i), and the other of the inverted repeats is 3′ of (i); (b) introducing into the mammalian, non-human embryonic cell a coding sequence encoding a transposase that acts on the inverted repeats to mediate nucleic acid transposition; (c) generating a transgenic mammal from the mammalian, non-human embryonic cell into which the nucleic acid construct and the coding sequence encoding the transposase have been introduced; and (d) administering to the transgenic mammal an inducing agent that induces expression of the polynucleotide from the inducible promoter.

In certain embodiments, the regulatory RNA is an shRNA. In one such embodiment, the shRNA is specific for an endogenous gene selected from (a) a gene encoding lipin, (b) a gene encoding VEGF, or (c) a gene that is an oncogene.

In certain embodiments, the inducible promoter comprises one or more TetO sequences, the nucleic acid construct further comprises a second transcription unit comprising a coding sequence encoding a TetR, and the one of the inverted repeats is 5′ of the second transcription unit, and the other of the inverted repeats is 3′ of the second transcription unit.

In certain embodiments, the pair of inverted repeats are derived from a piggyBac transposon, and the transposase is a piggyBac transposase. In one such embodiment, the one or the other of the inverted repeats comprises a polynucleotide sequence selected from SEQ ID NO:1, SEQ ID NO:2, SEQ ID NO:3, SEQ ID NO:4, and SEQ ID NO:5. In another such embodiment, the transposase comprises (a) an amino acid sequence having at least 90% amino acid sequence identity to SEQ ID NO:14, or (b) a fragment of (a).

In certain embodiments, the mammalian, non-human embryonic cell is a murine cell. In one such embodiment, the mammalian, non-human embryonic cell is a fertilized egg.

In yet another aspect, a nucleic acid construct is provided, wherein the nucleic acid construct comprises: (a) a first transcription unit comprising a polynucleotide operably linked to an inducible promoter, wherein the polynucleotide encodes a regulatory RNA specific for an endogenous gene; and (b) a pair of inverted repeats, wherein one of the inverted repeats is 5′ of (a), and the other of the inverted repeats is 3′ of (a).

In certain embodiments, the regulatory RNA is an shRNA. In one such embodiment, the shRNA is specific for an endogenous gene selected from (a) a gene encoding lipin, (b) a gene encoding VEGF, or (c) a gene that is an oncogene.

In certain embodiments, the inducible promoter comprises one or more TetO sequences, the nucleic acid construct further comprises a second transcription unit comprising a coding sequence encoding a TetR, and the one of the inverted repeats is 5′ of the second transcription unit, and the other of the inverted repeats is 3′ of the second transcription unit.

In certain embodiments, the inverted repeats are piggyBac inverted repeats. In certain embodiments, the one or the other of the inverted repeats comprises a polynucleotide sequence selected from SEQ ID NO:1, SEQ ID NO:2, SEQ ID NO:3, SEQ ID NO:4, and SEQ ID NO:5.

In certain embodiments, the second transcription unit further comprises a selectable marker. In one such embodiment, an IRES is disposed between the coding sequence encoding a TetR and the selectable marker. In certain embodiments, the coding sequence encoding a TetR is codon-optimized. In one such embodiment, the coding sequence encoding a TetR comprises the nucleic acid sequence of nucleotides 1-507 of SEQ ID NO:15.

In certain embodiment, the inducible promoter comprises an H1 or U6 promoter. In certain embodiments, the inducible promoter comprises at least two TetO sequences. In one such embodiment, the inducible promoter comprises the nucleic acid sequence of SEQ ID NO:16.

In yet another aspect, a cell comprising any of the above nucleic acid constructs is provided. In one such embodiment, the cell is a mammalian cell. In one such embodiment, the mammalian cell is an embryonic cell. In another such embodiment, the mammalian cell is a murine cell.

### BRIEF DESCRIPTION OF THE FIGURES

FIG. 1 shows a piggyBac-based vector, “PB(luc-shRNA),” for inducible expression of shRNA specific for luciferase, as described in Example A.

FIG. 2 shows the nucleotide sequence (SEQ ID NO:17) of the vector of FIG. 1, with functional elements annotated as described in Example A. The amino acid sequence (SEQ ID NO:21) of a codon-optimized TetR is also shown. The amino acid sequence (SEQ ID NO:22) of a puromycin selectable marker is also shown.

FIG. 3 shows doxycycline (Dox)-induced expression of shRNA specific for luciferase and knock down of luciferase activity in embryonic stem (ES) cells transfected with PB(luc-shRNA), as described in Example B.

FIG. 4 shows Dox-induced expression of shRNA specific for luciferase and knock down of luciferase activity in clones isolated from ES cells transfected with PB(luc-shRNA), as described in Example B.

FIG. 5 shows quantification of bioluminescence from the ES cells in FIG. 4.

FIG. 6 shows quantification of bioluminescence from embryoid bodies derived from ES cells transfected with PB(luc-shRNA) and treated with Dox, as described in Example B.

FIG. 7 shows the effects of Dox administration for three days to luciferase-expressing transgenic mice derived from single cell embryos injected with PB(luc-shRNA), as described in Example C.

FIG. 8 shows quantification of bioluminescence of the transgenic mice in FIG. 7, as described in Example C.

FIG. 9 shows the effect of Dox administration for seven days to a luciferase-expressing transgenic mouse derived from a single cell embryo injected with PB(luc-shRNA), as described in Example C.

FIG. 10 shows a strategy for constructing a piggyBac based vector for inducible expression of shRNA specific for lipin, as described in Example D.

FIG. 11 shows the nucleotide sequence (SEQ ID NO:18) of pCAG-PBase, as described in Example C.

### DETAILED DESCRIPTION OF EMBODIMENTS

Compositions and methods are provided herein for the expression of nucleic acids. In certain embodiments, such compositions and methods allow for inducible expression of nucleic acids in transgenic cells and animals from transposon-based nucleic acid constructs. In certain embodiments, such compositions and methods may be used to modulate gene expression in an inducible manner. In additional embodiments, such compositions and methods may be used to inhibit, or “knock down” expression of a nucleic acid sequence, e.g., an endogenous gene, in an inducible manner, making it possible to create “conditional knock downs” of genes, e.g., genes whose disruption by conventional gene targeting techniques would otherwise cause early-stage lethality.

### I. DEFINITIONS

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “isolated,” with reference to a cell or biological molecule, such as a nucleic acid, polypeptide, or antibody, is one which has been identified and separated and/or recovered from at least one component of its natural environment.

The term “stringent conditions,” with respect to hybridization conditions, means that hybridization of nucleic acids takes place in 5×SSC, 5×Denhardt solution, 1% SDS, and 100 μg/ml denatured salmon sperm DNA at 65° C.; and hybridization is followed by the following washes (the second wash being a high stringency wash): 10 min in 2×SSC containing 0.1% SDS at room temperature; and 30 min in 0.1×SSC containing 0.1% SDS at 65° C. See Ausubel et al., Current Protocols in Molecular Biology (1995) Wiley Interscience Publishers for further details.

The term “nucleic acid construct” refers to a recombinant nucleic acid molecule comprising polynucleotide segments not normally associated with one another in nature. A nucleic acid construct may be extrachromosomal or integrated into a host cell's chromosome.

The term “polynucleotide of interest” is non-limiting and refers to any polynucleotide. The term “flank” means that a given nucleic acid sequence(s) appears 5′ and 3′ of a particular reference sequence. Intervening sequences may occur between the given nucleic acid sequence(s) and the reference sequence.

The term “transcription unit” refers to a region within a nucleic acid construct that comprises at least one polynucleotide sequence to be transcribed, wherein the sequence(s) is operably linked to a particular promoter.

The term “siRNA” or “short interfering RNA” refers to a double stranded RNA that has the ability to reduce or inhibit expression of a target polynucleotide when the siRNA is expressed in the same cell as the target polynucleotide. The complementary strands of an siRNA that form the double stranded RNA typically have substantial or complete identity. In one embodiment, an siRNA refers to a double-stranded RNA, one strand of which (also referred to as the “antisense” strand) has substantial or complete identity to at least a portion of a target mRNA. In certain embodiments, an siRNA is about 15-50 nucleotides in length, about 20-30 nucleotides in length, about 20-25 nucleotides in length, or 24-29 nucleotides in length, including any length that is an integer within the above-stated ranges. See also PCT/US03/07237, published as WO03076592, herein incorporated by reference in its entirety. An siRNA molecule is “specific” for a target polynucleotide if it (a) selectively binds to the target polynucleotide (or to an mRNA transcribed from the target polynucleotide, if the target polynucleotide is a gene) and/or (b) reduces expression of the target polynucleotide by at least about 10%, 20%, 30%, 40%, 50%, 60%, 70%, 80%, 90%, 91%, 92%, 93%, 94%, 95%, 96%, 97%, 98%, or 99% when the siRNA is expressed in a cell that expresses the target polynucleotide.

The term “siRNA” encompasses RNA capable of forming a hairpin structure, e.g., microRNA precursors (pre-miRNA) and short hairpin RNA (shRNA). See, e.g., Brummelkamp et al. (2002)Science 550-553. A pre-miRNA or an shRNA is a self-complementary RNA molecule having a sense region, an antisense region, and a loop region, and which is capable of forming a hairpin structure. In certain embodiments, the sense and antisense regions are each about 15-50 nucleotides in length, about 20-30 nucleotides in length, about 20-25 nucleotides in length, or about 24-29 nucleotides in length, including any length that is an integer within the foregoing ranges; and the loop portion is about 2-15 nucleotides in length or about 6-9 nucleotides in length, including any length that is an integer within the foregoing ranges.

The term “RNAi” or RNA interference” refers to partial or complete inhibition of gene expression by an RNA-mediated mechanism, e.g., by a double-stranded RNA-mediated mechanism.

The term “regulatory RNA” or “regulatory RNA molecule” refers to an RNA capable of regulating expression of a gene, e.g., by regulating expression of the corresponding mRNA. Such regulatory RNAs include, but are not limited to, RNA capable of RNAi. An regulatory RNA is “specific” for a target polynucleotide if it (a) selectively binds to the target polynucleotide (or to an mRNA transcribed from the target polynucleotide, if the target polynucleotide is a gene) and/or (b) reduces expression of the target polynucleotide by at least about 10%, 20%, 30%, 40%, 50%, 60%, 70%, 80%, 90%, 91%, 92%, 93%, 94%, 95%, 96%, 97%, 98%, or 99% when the regulatory RNA is expressed in a cell that expresses the target polynucleotide.

The term “regulatory element” refers to one or more nucleotide sequences that modulate transcription and/or translation of a nucleotide sequence. Transcriptional regulatory elements include, but are not limited to, a promoter capable of driving expression of an operably linked polynucleotide; an operator sequence within a promoter that influences the transcription-promoting activity of a promoter; a transcription termination sequence; and a polyadenylation signal sequence.

The term “operably linked” refers to a juxtaposition of two or more components, wherein the components are in a relationship that permits them to function in their intended manner. For example, a promoter is “operably linked” to a polynucleotide sequence if it acts in cis to control the transcription of the polynucleotide sequence. Nucleic acid sequences that are “operably linked” may or may not be contiguous.

The term “expression” as used herein refers to transcription or translation of a given nucleic acid that occurs within a cell. The level of expression may be determined, e.g., on the basis of either the amount of RNA that is transcribed from the nucleic acid, or, if the RNA is translated, the amount of encoded protein. For example, mRNA transcribed from a given nucleic acid can be quantified by PCR or by northern hybridization (see Sambrook et al., Molecular Cloning: A Laboratory Manual, Cold Spring Harbor Laboratory Press (1989)). Protein encoded by a given nucleic acid can be quantified by various methods, e.g., by ELISA, by assaying for the biological activity of the protein, or by employing assays that are independent of such activity, such as western blotting or radioimmunoassay, using antibodies that are recognize and bind to the protein. See Sambrook et al., 1989, supra.

The term “inhibit” means to partially or completely reduce or block a particular process or result.

The term “promoter” refers to a polynucleotide sequence that controls transcription of a nucleic acid to which it is operably linked. A promoter includes signals for RNA polymerase binding and transcription initiation. In some embodiments, a promoter may comprise additional regulatory elements, e.g., operator sequences. A large number of promoters including constitutive, inducible and repressible promoters from a variety of different sources, are well known in the art (and identified in databases such as GenBank) and are available as or within cloned polynucleotides (from, e.g., depositiories such as ATCC as well as other commercial or individual sources). With inducible promoters, the activity of the promoter increases or decreases in response to a signal, e.g., an inducing agent. Among the promoters that have been identified as strong promoters are the SV40 early promoter, adenovirus major late promoter, mouse metallothionein-I promoter, Rous sarcoma virus long terminal repeat, and human cytomegalovirus immediate early promoter (CMV).

The term “inducible promoter” refers to a promoter whose activity can be regulated by adding or removing one or more specific signals. For example, an inducible promoter may activate transcription of an operably linked nucleic acid under a specific set of conditions, e.g., in the presence of an inducing agent that activates the promoter and/or relieves repression of the promoter.

The term “inducing agent” refers to any agent capable of regulating the activity of an inducible promoter. Inducing agents include, but are not limited to, chemical compounds, biological macromolecules, or any combination thereof.

The term “inverted repeat” or “IR” refers to a nucleic acid sequence derived from a transposon and acted upon by a transposase, wherein two copies of the nucleic acid sequence are in the opposite orientation when present in a transposable nucleic acid molecule. Inverted repeat sequences may be imperfect, meaning that the nucleic acid sequences are not perfect copies of each other, so long as the inverted repeats are capable of mediating transposition of a polynucleotide located between the inverted repeats.

The term “piggyBac” refers to a family of transposons initially identified in the Lepidopteran Trichopulsia ni, wherein the transposon is related to Class II DNA transposable elements. A piggyBac transposon has been previously described in the art as “IFP2.” See Cary et al. (1989) Virology 172:156-169.

The term “Class II IR” or “Class II inverted repeat” refers to an inverted repeat derived from a Class II DNA transposable element and acted upon by a Class II transposase.

The term “piggyBac inverted repeat”or “piggyBac IR” refers to an inverted repeat derived from a piggyBac transposon and acted upon by a piggyBac transposase.

The term “internal ribosome entry site” or “IRES” describes a polynucleotide sequence which promotes translation initiation and allows two cistrons (open reading frames) to be translated from a single transcript in an animal cell. The IRES provides a ribosome entry site for translation of an open reading frame operably linked to the IRES. Unlike bacterial mRNA which can be polycistronic (i.e., can encode several different polypeptides from a single mRNA), most mRNAs of animal cells are monocistronic and code for the synthesis of only one protein. When a polycistronic transcript is present in a eukaryotic cell, translation generally initiates from the 5′ most translation initiation site and terminates at the first stop codon. The transcript is then released from the ribosome, resulting in the translation of only the first encoded polypeptide in the polycistronic transcript. In a eukaryotic cell, a polycistronic transcript having an IRES operably linked to a second or subsequent open reading frame in the transcript allows for the translation of that open reading frame to produce two or more polypeptides encoded by the same transcript. The use of IRES elements in vector construction has been previously described, see, e.g., Pelletier et al., Nature 334: 320-325 (1988); Jong et al., J. Virol. 63: 1651-1660 (1989); Davies et al., J. Virol. 66: 1924-1932 (1992); Adam et al. J. Virol. 65: 4985-4990 (1991); Morgan et al. Nucl. Acids Res. 20: 1293-1299 (1992); Sugimoto et al. Biotechnology 12: 694-698 (1994); Ramesh et al. Nucl. Acids Res. 24: 2697-2700 (1996).

The term “selectable marker” refers to a polynucleotide that allows cells carrying the polynucleotide to be specifically selected for or against, in the presence of a corresponding selection agent. By way of illustration, an antibiotic resistance gene can be used as a positive selectable marker that allows the host cell transformed with the gene to be positively selected for in the presence of the corresponding antibiotic; a non-transformed host cell would not be capable of sustained growth or survival under selection conditions. Selectable markers can be positive, negative or bifunctional. Positive selectable markers allow selection for cells carrying the marker, whereas negative selection markers allow cells carrying the marker to be selectively eliminated. In certain embodiments, a selectable marker will confer resistance to a drug or compensate for a metabolic or catabolic defect in the host cell. Selectable markers include amplifiable selectable genes, and include variants, fragments, functional equivalents, derivatives, homologs and fusions of a native selectable marker so long as the encoded product retains the selectable property. Useful derivatives generally have substantial sequence similarity (at the amino acid level) in regions or domains of the selectable marker associated with the selectable property. A variety of selectable markers have been described, including bifunctional (i.e., positive/negative) markers (see e.g., WO 92/08796, published 29 May 1992, and WO 94/28143, published 8 Dec. 1994), incorporated by reference herein. For example, selectable markers commonly used with eukaryotic cells include the genes for aminoglycoside phosphotransferase (APH), hygromycin phosphotransferase (hyg), dihydrofolate reductase (DHFR), thymidine kinase (tk), glutamine synthetase, asparagine synthetase, and genes encoding resistance to neomycin (G418), puromycin, histidinol D, bleomycin and phleomycin.

The term “introducing,” “introduced,” and grammatical variants thereof, with reference to transfer of nucleic acids, refers to human intervention that either directly or indirectly results in the introduction of a nucleic acid into a cell. For example, a nucleic acid may be directly introduced into a cell, for example, by transfection, and that nucleic acid is also considered to have been “introduced” into any of the cell's progeny that contain it.

The term “polypeptide” or “protein,” as used interchangeably herein, refer to polymers of amino acids of any length. The term also includes proteins that are post-translationally modified through reactions that include glycosylation, acetylation and phosphorylation. The term “peptide” refers to short polypeptides that are generally less than about 30 amino acids in length.

The term “codon-optimized” refers to a nucleic acid coding sequence that has been adapted for expression in the cells of a given vertebrate by replacing one or more codons with one or more codons that are more frequently used in the translation of nucleic acids in that vertebrate.

The term “TetO” or “TetO sequence” refers to a Tet operator sequence that is capable of binding TetR.

The term “TetR” refers to a wild-type Tet repressor or variant thereof capable of binding one or more TetO sequences.

The term “substantially similar” or “substantially the same,” as used herein, denotes a sufficiently high degree of similarity between two numeric values (for example, expression levels of TetR), such that one of skill in the art would consider the difference between the two values to be of little or no biological and/or statistical significance within the context of the biological characteristic measured by said values.

The term “mammal” refers to any animal classified as a mammal, including farm animals (such as cows), sport animals, pets (such as cats, dogs, and horses), primates (including human and non-human primates), and rodents (e.g., mice and rats). In certain embodiments, a mammal is a human.

The term “transgenic” is used herein to describe the property of harboring a transgene. For instance, a “transgenic organism” is any animal, including mammals, fish, birds and amphibians, in which one or more of the cells of the animal contain nucleic acid introduced by way of human intervention, such as by the methods described herein. In a transgenic animal comprising a transgene that encodes a polypeptide of interest, for example, the transgene typically will direct cell(s) of the transgenic animal to express or overexpress the polypeptide. However, according to some embodiments of the invention, expression of a regulatory RNA can be used to down regulate the expression of a particular endogenous gene through antisense or RNA interference mechanisms.

The term “percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “percent (%) nucleic acid sequence identity” with respect to a reference polynucleotide sequence is defined as the percentage of nucleotides in a candidate sequence that are identical with the nucleotides in the reference polynucleotide sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % nucleic acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % amino acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. Unless specifically stated otherwise, all % nucleic acid sequence identity values used herein are obtained as described in the immediately preceding paragraph using the ALIGN-2 computer program.

### II. EMBODIMENTS OF THE INVENTION

Compositions and methods are provided herein for the expression of nucleic acids. In certain embodiments, such compositions and methods allow for inducible expression of nucleic acids in transgenic cells and animals. In certain embodiments, such compositions include transposon-based nucleic acid constructs. In certain embodiments, such compositions and methods may be used to modulate endogenous gene expression. In additional embodiments, such compositions and methods may be used to inhibit, or “knock down” expression of a nucleic acid sequence, e.g., an endogenous gene in an inducible manner, making it possible to create “conditional knock-downs” of genes, e.g., genes whose disruption by conventional gene targeting techniques would otherwise cause early-stage lethality.

A. Compositions

In one aspect, nucleic acid constructs are provided for expression of nucleic acids. In one embodiment, a nucleic acid construct comprises 1) a transcription unit comprising at least one polynucleotide of interest operably linked to an inducible promoter and (2) transposon-derived inverted repeats flanking the polynucleotide of interest. The components of such nucleic acid constructs are further described in the embodiments below:

1. Components

a) Inducible Promoter Systems

In one aspect, an inducible promoter system is used to regulate the expression of a polynucleotide of interest. In various embodiments of an inducible promoter system, a polynucleotide of interest is operably linked to an inducible promoter. Transcription of a polynucleotide of interest from an inducible promoter may be activated, e.g., by an inducing agent. In one such embodiment, an inducible promoter is inactive or has low basal activity in the absence of an inducing agent, and is active in the presence of an inducing agent. Transcription in the presence of an inducing agent may be 5-, 10-, 50, 100, or 500-fold greater than transcription in the absence of an inducing agent.

An inducing agent may act directly on a promoter, e.g., by binding to a promoter and activating transcription from the promoter. Examples of such inducing agents include, but are not limited to, heavy metal ions, interferon, and glucocorticoid, described below in Table 1. Alternatively, an inducing agent may act indirectly on a promoter, e.g., by acting through a polypeptide that influences promoter activity. For example, in one embodiment, an inducing agent activates (e.g., by binding to) a polypeptide, such as a receptor, and the activated polypeptide then activates transcription from a promoter. Examples of such inducing agents include, but are not limited to, ecdysone, RU486, and estrogen, described below in Table 1. Other inducing agents may include a specified growth condition, e.g., a “heat shock.” In another embodiment, an inducing agent deactivates a polypeptide that represses the promoter, thereby activating transcription by relieving repression. Examples of such inducing agents include, but are not limited, to IPTG (for use in a Lac expression system) and tetracycline and its analogs (for use in a Tet expression system).

Exemplary inducible promoter systems for use in eukaryotic cells include, but are not limited to, hormone-regulated elements (e.g., see Mader, S, and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. et al. (1993) Biochemistry 32:10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci. 21:377-405, and are provided in Table 1 below.

TABLE 1

| Inducible Promoter | Promoter/regulatory | |
|---|---|---|
| System | elements | Inducing Agent |
| heat shock system | heat shock promoter | Temperature shift, typically |
| | | from 37 to about 42° C. |
| heavy metal ion | metallothionein gene | heavy metal ion, e.g., |
| system | promoter comprising | Cd2+, Zn2+ |
| | metal responsive | |
| | elements (MREs) | |
| interferon system | MX1 promoter | Interferon or analogs |
| | comprising interferon | |
| | responsive element | |
| Glucocorticoid | promoter comprising | Glucocorticoid or analogs |
| system | GREs (glucocorticoid | |
| | responsive elements) | |
| Estrogen system | promoter comprising | Estrogen or analogs, which |
| | GAL4 responsive | act through a Gal4- |
| | element(s) | mammalian estrogen |
| | | receptor fusion protein |
| | | (with optional VP16 |
| | | transactivation domain) |
| RU486 system | promoter comprising | RU486 or analogs, which |
| | GAL4 responsive | act through a Gal4- |
| | element(s) | modified progesterone |
| | | receptor fusion protein, |
| | | with optional VP16 |
| | | transactivation domain |
| Ecdysone system | promoter comprising | Ecdysone or analogs (e.g., |
| | ecdysone responsive | muristerone), which act |
| | element(s) | through ecdysone receptor, |
| | | preferably fused to VP16 |
| | | transactivation domain |
| Lac system | promoter comprising | IPTG or other lactose |
| | one or more lac | analogs, which act by |
| | operators (lacO) | relieving repression of |
| | | the promoter by Lac |
| | | repressor (LacR) |
| Tet system | Promoter comprising | Tetracycline or |
| | one or more Tet | derivatives/analogs (e.g., |
| | operators (TetO) | anhydrotetracycline, |
| | | doxycycline) |

An exemplary inducible promoter system for use in the present invention is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Codon usage tables are readily available. See Nakamura, Y., et al. Nucl. Acids Res. (2000) 28:292. By utilizing these or similar tables, one of ordinary skill in the art can apply codon usage frequencies to any given polypeptide sequence in order to design a codon-optimized nucleic acid encoding the polypeptide. Codon-optimized coding regions can be designed by various different methods known in the art, some of which are described herein and in US Patent Application publication No. 20040209241.

In one aspect, use of a codon-optimized TetR allows for tighter control of inducible polynucleotide expression, e.g., by (1) increasing TetR expression, (2) allowing for induction of expression using lower levels of an inducing agent, and/or (3) minimizing “leaky” expression of a polynucleotide of interest in the absence of an inducing agent. A codon-optimized TetR is described in detail in co-pending U.S. application Ser. No. 11/460,606, filed Jul. 27, 2006, which is expressly incorporated by reference herein in its entirety. The sequence of wild-type Tet repressor protein is known in the art (see, e.g., GenBank Accession No. J01830). Assays for testing TetR protein binding to TetO sequences are described in Lederer et al (1995) Anal. Biochemistry 232:190-196

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli. (See, Brown et al., Cell 49:603-612 (1987). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

Various promoters may be used in nucleic acid constructs of the invention, including synthetic promoters and native promoters of either prokaryotic or eukaryotic origin. In certain embodiments, a variety of RNA polymerase III (pol III) promoters can be used, e.g., pol III promoters derived from any mammal, such as human or mouse. Such pol III promoters include, but are not limited to, promoters derived from H1 RNA or U6 snRNA genes, referred to herein as “H1 promoter” or “U6 promoter,” respectively. Description of other pol III promoters can be found, e.g., in Paule and White, Nuc. Acids Res. (2000) 28:1283-1298, which is hereby incorporated by reference in its entirety. In certain embodiments, a variety of RNA polymerase II (pol II) promoters can be used, including for example, the CMV promoter. A pol II promoter can be a ubiquitous promoter capable of driving expression in many tissues, for example, the Ubiquitin-C promoter, CMV promoter, beta-actin promoter or PGK promoter. In other embodiments, a pol II promoter is a tissue- or cell type-specific promoter or developmental stage-specific promoter.

Other promoters useful in nucleic acid constructs of the invention include viral promoters (e.g., Rous Sarcoma virus long terminal repeat promoter (pRSV); promoters from polyoma virus, fowlpox virus (UK 2,211,504 published 5 Jul. 1989), adenovirus (such as Adenovirus 2 or 5), herpes simplex virus (thymidine kinase promoter), bovine papilloma virus, avian sarcoma virus, cytomegalovirus (CMV), a retrovirus (e.g., MoMLV, or RSV LTR), Hepatitis-B virus, myeloproliferative sarcoma virus (MPSV), VISNA, and Simian Virus 40 (SV40); and the SP6, T3 and T7 promoters); immunoglobulin promoters; heat-shock promoters; or metallothionein promoters. The early and late promoters of the SV40 virus may be conveniently obtained as a restriction fragment that also contains the SV40 viral origin of replication. Fiers et al., Nature, 273:113 (1978); Mulligan and Berg, Science, 209:1422-1427 (1980); Pavlakis et al., Proc. Natl. Acad. Sci. USA, 78:7398-7402 (1981). The immediate early promoter of the human cytomegalovirus (CMV) may be conveniently obtained as a Hind III E restriction fragment. Greenaway et al., Gene, 18:355-360 (1982).

It is further contemplated that an inducible expression system can incorporate a recombination system, for example, the Cre/lox system of bacteriophage Pi, the FLP/FRT system of the yeast 2 uM plasmid, the 1/RS system of the yeast plasmid pSR1, or the modified Gin/gix system of bacteriophage Mu. In a particular embodiment, an inducible expression system incorporates the Cre/loxP recombination system. Briefly, Cre is a 38 kDa recombinase protein from bacteriophage Pi which mediates intramolecular (excisive or inversional) and intermolecular (integrative) site specific recombination between loxP sites as described by Sauer (1993) Methods Enzymol. 225:890-900, which is incorporated herein by reference. A loxP site (“locus of crossing over” site) consists of two 13 by inverted repeats separated by an 8 by asymmetric spacer region. One molecule of Cre binds per inverted repeat or two Cre molecules line up at a given loxP site. Recombination occurs in the 8 base pair asymmetric spacer region, which also is responsible for the directionality of the site. Two loxP sites in opposite orientation to each other invert the intervening piece of DNA; two sites in the same orientation dictate excision of the intervening DNA between the sites leaving one loxP site behind.

The ability to excise a nucleic acid sequence at a particular time can be exploited by flanking a nucleic acid sequence with a pair of lox P sites and introducing the recombinase when excision is desired. If desired, a Cre-expressing transgene can be placed under control of an inducible and/or tissue-specific promoter to allow excision of a nucleic acid sequence in selected cells and at selected times. In one embodiment of an inducible expression system, a polynucleotide comprising a “stuffer fragment” (further described below) is located within a promoter or between a promoter and a nucleic acid sequence for which inducible expression is desired (“inducible sequence”). The stuffer fragment is flanked by loxP sites, so that a Cre-mediated recombination event leads to excision of the stuffer fragment and juxtaposition of the inducible sequence and the promoter, such that the inducible sequence and the promoter are operably linked.

A “stuffer fragment” refers to a polynucleotide that is inserted into a promoter or between a promoter and an inducible sequence, and that comprises a transcription stop signal specific to the promoter. The presence of the stuffer fragment thus prevents transcription of the inducible sequence from the promoter and keeps the promoter-inducible sequence transcription unit in an inactive state. Upon addition of a recombinase enzyme (as described above), site-specific excision of the stuffer fragment containing the promoter-specific transcription stop signal results in juxtaposition of the promoter and the inducible sequence, which in turn results in transcription of the inducible sequence.

A stuffer fragment can be of any nucleotide sequence and preferably is a sequence that is not prone to conformational changes. For example, a stuffer fragment can be a segment of the lacZ gene or any other desired nucleic acid segment provided that it comprises a transcription stop signal that is functional in preventing transcription. If desired, the stuffer fragment can contain additional features, for example, a selectable marker that allows for easy detection and determination of the transcriptional state as induced versus non-induced.

The size of a stuffer fragment can be 500 base pairs or more, 600 base pairs or more, 700 base pairs or more, 800 base pairs or more, 1000 base pairs or more, 1200 base pairs or more, or 1400 base pairs or more, so long as it is capable of (a) inhibiting transcription and/or (b) being excised in an enzyme-mediated recombination event. An example of a stuffer fragment is a 1 kb segment of the lacZ gene that contains a sequence consisting of five adjacent thymines corresponding to a murine U6 promoter specific transcription stop signal.

b) Transposon-Based Systems

A suitable transposon-based system may be used to create transgenic cells expressing a polynucleotide of interest. In one embodiment, a suitable transposon-based system comprises (1) a nucleic acid comprising the polynucleotide of interest flanked by transposon inverted repeats that allow for excision and transposition of the nucleic acid; and (2) a transposase that acts upon the inverted repeats to mediate transposition of the nucleic acid. In one embodiment, inverted repeats and the transposases that act on them are derived from a Class II transposable element, including but not limited to, piggyBac, tagalong, hobo, hermes, Ac, and Tam3 transposable elements.

In one embodiment, a nucleic acid comprising a polynucleotide of interest is flanked by transposon inverted repeats. In one such embodiment, the inverted repeats are on the 5′ and 3′ ends of the nucleic acid comprising the polynucleotide of interest.

In one embodiment, inverted repeats allow for transposition of a polynucleotide of interest into a vertebrate genome, such as a mammalian genome. Such inverted repeats include, but are not limited to, piggyBac inverted repeats or inverted repeats from a transposon of the Tc1/mariner transposon superfamily. That superfamily includes, but is not limited to, the “Sleeping Beauty” and “Frog Prince” transposons.

piggyBac transposons were initially identified in the Lepidopteran Trichopulsia ni. See Cary et al. (1989) Virology 172:156-169. In Trichopulsia ni, piggyBac is a 2475 bp short inverted repeat element comprising an open reading frame of 2.1 kb that encodes a functional transposase. piggyBac transposes via a cut-and-paste mechanism, inserting at 5′TTAA3′ target sites that are duplicated upon insertion and excising precisely, leaving no footprint. piggyBac's inverted repeat elements and their ability to drive transposition have been characterized. See, e.g., U.S. Pat. Nos. 6,218,185, and 6,962,810, which are expressly incorporated by reference herein.

In one embodiment, inverted repeats are derived from the piggyBac transposon. In one such embodiment, an inverted repeat comprises the nucleic acid sequence of

| | |
|---|---|
| 5′CCCTAGAAAGATA3′. | (SEQ ID NO: 1) |

In another of such embodiments, an inverted repeat comprises the nucleic acid sequence of

(SEQ ID NO: 2)

5′ CCCTAGAAAGATAGTCTGCGTAAAATTGACGCATG 3′,

or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:2, wherein such inverted repeat is capable of mediating transposition of an operably linked nucleic acid. In another of such embodiments, an inverted repeat comprises the nucleic acid sequence of

(SEQ ID NO: 3)

5′ CCCTAGAAAGATAATCATATTGTGACGTACGTTAAAGATAATCATGC
GTAAAATTGACGCATG 3′,

or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:3, wherein such inverted repeat is capable of mediating transposition of an operably linked nucleic acid.

In a particular embodiment, a nucleic acid capable of transposition comprises a polynucleotide of interest flanked by (1) a first inverted repeat comprising SEQ ID NO:2, or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:2; and (2) a second inverted repeat comprising the reverse complement of SEQ ID NO:3 (i.e., SEQ ID NO:4), or a nucleic acid sequence having at least 80%, 85%, 90%, 91%, 92%, 93%, 94%, 95%, 96%, 97%, 98%, 99% identity to SEQ ID NO:4, or a nucleic acid that hybridizes under stringent conditions to the complement of SEQ ID NO:4.

In another particular embodiment, a nucleic acid capable of transposition comprises a polynucleotide of interest flanked by (1) a first inverted repeat comprising SEQ ID NO:3, or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:3; and (2) a second inverted repeat comprising the reverse complement of SEQ ID NO:2 (i.e., SEQ ID NO:5), or a nucleic acid sequence having at least 80%, 85%, 90%, 91%, 92%, 93%, 94%, 95%, 96%, 97%, 98%, 99% identity to SEQ ID NO:5, or a nucleic acid that hybridizes under stringent conditions to the complement of SEQ ID NO:5.

The first and second inverted repeats may be at the 5′ and 3′ ends of the polynucleotide of interest, respectively. Alternatively, the first and second inverted repeats may be at the 3′ and 5′ ends of the polynucleotide of interest, respectively. Exemplary configurations (each depicting a single nucleic acid molecule) are as follows:

Configuration 1

(SEQ ID NO: 2)

5′ CCCTAGAAAGATAGTCTGCGTAAAATTGACGCATG

polynucleotide of interest—

(SEQ ID NO: 4)

CATGCGTCAATTTTACGCATGATTATCTTTAACGTACGTCACAATATG
ATTATCTTTCTAGGG 3′,

or

Configuration 2

(SEQ ID NO: 3)

5′ TCATATTGTGACGTACGTTAAAGATAATCATGCGTAAAATTGACG
CATG

—polynucleotide of interest—

(SEQ ID NO: 5)

CATGCGTCAATTTTACGCAGACTATCTTTCTAGGG 3′

In certain embodiments, inverted repeats are derived from the Sleeping Beauty transposon. Such inverted repeats are described, e.g., in U.S. Pat. Nos. 6,613,752 and 6,489,458, which are expressly incorporated by reference herein. In one of such embodiments, an inverted repeat comprises a nucleic acid sequence selected from:

(SEQ ID NO: 6)

5′ GTTCAAGTCG GAAGTTTACA TACACTTAG 3′

(SEQ ID NO: 7)

5′ CAGTGGGTCA GAAGTTTACA TACACTAAGG 3′

(SEQ ID NO: 8)

5′ CAGTGGGTCA GAAGTTAACA TACACTCAAT T 3′

(SEQ ID NO: 9)

5′ AGTTGAATCG GAAGTTTACA TACACCTTAG 3′

In another of such embodiments, an inverted repeat comprises the nucleic acid sequence of:

(SEQ ID NO: 10)

5′ AGTTGAAGTC GGAAGTTTAC ATACACTTAA GTTGGAGTCA
TTAAAACTCG TTTTTCAACT ACACCACAAA TTTCTTGTTA
ACAAACAATA GTTTTGGCAA GTCAGTTAGG ACATCTACTT
TGTGCATGAC ACAAGTCATT TTTCCAACAA TTGTTTACAG
ACAGATTATT TCACTTATAA TTCACTGTAT CACAATTCCA
GTGGGTCAGA AGTTTACATA CACTAA 3′,

or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:10, wherein such inverted repeat is capable of mediating transposition of an operably linked nucleic acid. In another of such embodiments, an inverted repeat comprises the nucleic acid sequence of:

(SEQ ID NO: 11)

5′TTGAGTGTAT GTTAACTTCT GACCCACTGG GAATGTGATG
AAAGAAATAA AAGCTGAAAT GAATCATTCT CTCTACTATT
ATTCTGATAT TTCACATTCT TAAAATAAAG TGGTGATCCT
AACTGACCTT AAGACAGGGA ATCTTTACTC GGATTAAATG
TCAGGAATTG TGAAAAAGTG AGTTTAATG TATTTGGCTA
AGGTGTATGT AAACTTCCGA CTTCAACTG 3′,

or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:11, wherein such inverted repeat is capable of mediating transposition of an operably linked nucleic acid.

In certain embodiments, inverted repeats are derived from the Frog Prince transposon. Such inverted repeats are described, e.g., in U.S. Patent Application Publication No. US 2005/0241007 A1, which is expressly incorporated by reference herein. In one of such embodiments, an inverted repeat comprises the nucleic acid sequence of:

| | |
|---|---|
| 5′ TGTG AAAAAGTGTT TGCCCCC 3′ | (SEQ ID NO: 12) |

In another of such embodiments, an inverted repeat comprises the nucleic acid sequence of:

(SEQ ID NO: 13)

5′ CAGTGGTGTG AAAAAGTGTT TGCCCCCTTC CTCATTTCCT
GTTCCTTTGC ATGTTTGTCA CACTTAAGTG TTTCGGAACA
TCAAACCAAT TTAAACAATA GTCAAGGACA ACACAAGTAA
ACACAAAATG CAATTTGTAA ATGAAGGTGT TTATTATTAA
AGGTGAAAAA AAATCCAAAC CATCATGGCC CTGTGTGAAA
AAGTGATTGC CCCCCTTGTT AAAACATACT ATAACTGTGG
TTGTCCACAC 3′

or a nucleic acid sequence having at least 80%, 85%, 90%, 91%, 92%, 93%, 94%, 95%, 96%, 97%, 98%, 99% identity thereto, or a nucleic acid that hybridizes under stringent conditions to the complement of SEQ ID NO:13, wherein such inverted repeat is capable of mediating transposition of an operably linked nucleic acid.

Transposases that act on any of the above-described inverted repeats are provided herein. In one embodiment, a piggyBac, Sleeping Beauty, or Frog Prince transposase is provided. Such transposases are described, e.g., in the above-cited publications. A transposase may be a naturally occurring transposase or an active fragment or variant thereof, e.g., a variant having at least 80%, 85%, 90%, 91%, 92%, 93%, 94%, 95%, 96%, 97%, 98%, 99% amino acid sequence identity to a naturally occurring transposase. A transposase may also be an engineered transposase. A transposase may be derived from any source so long as it is active, i.e., capable of acting upon inverted repeats to mediate nucleic acid transposition. A transposase or a nucleic acid encoding a transposase may be introduced into a cell before, after, or concurrently with introduction of a nucleic acid comprising a polynucleotide of interest flanked by inverted repeats. A nucleic acid encoding a transposase and a polynucleotide of interest flanked by inverted repeats may be present on the same or separate nucleic acid constructs. Transcription of a nucleic acid encoding a transposase may be driven by any of the promoters (e.g., constitutive, inducible, or bifunctional) discussed herein.

In a particular embodiment, a piggyBac transposase (i.e., a transposase derived from a piggyBac transposon) is provided. In one such embodiment, a piggyBac transposase comprises the following amino acid sequence from the piggyBac transposon of the Lepidopteran Trichopulsia ni:

| (SEQ ID NO: 14) | |
|---|---|
| 1 | mgsslddehi lsallqsdde lvgedsdsei sdhvseddvq sdteeafide vhevqptssg |
| 61 | seildeqnvi eqpgsslasn kiltlpqrti rgknkhcwst skstrrsrvs alnivrsqrg |
| 121 | ptrmcrniyd pllcfklfft deiiseivkw tnaeislkrr esmtgatfrd tnedeiyaff |
| 181 | gilvmtavrk dnhmstddlf drslsmvyvs vmsrdrfdfl irclrmddks irptlrendv |
| 241 | ftpvrkiwdl fihqciqnyt pgahltideq llgfrgrcpf rmyipnkpsk ygikilmmcd |
| 301 | sgtkymingm pylgrgtqtn gvplgeyyvk elskpvrgsc rnitcdnwft siplaknllq |
| 361 | epykltivgt vrsnkreipe vlknsrsrpv gtsmfcfdgp ltlvsykpkp akmvyllssc |
| 421 | dedasinest gkpqmvmyyn qtkggvdtld qmcsvmtcsr ktnrwpmall ygminiacin |
| 481 | sfiiyshnvs skgekvqsre kfmrnlymsl tssfmrkrle aptlkrylrd nisnilpnev |
| 541 | pgtsddstee pvtkkrtyct ycpskirrka nasckkckkv icrehnidmc qscf, |

or an active fragment or variant thereof. Such fragments and variants include, but are not limited to, those described in Zimowska et al. (2006) Insect Biochem. Mol. Biol. 36(5):421-428, and in NCBI Accession Nos. ABC88680.1, ABC88678.1, ABC88677.1, ABC88675.1, ABC88671.1, and AAE68098.1, which are hereby incorporated by reference.

c) Polynucleotide of Interest

In the nucleic acid constructs of the invention, a polynucleotide of interest whose expression is under control of an inducible promoter is not limiting. For example, a polynucleotide of interest may or may not encode a polypeptide.

In one embodiment, a polynucleotide of interest encodes a polypeptide whose transgenic expression is desired, e.g., to observe the phenotypic impact of such transgenic expression and/or for “rescue” experiments in which endogenous expression of the polypeptide or functional equivalent is absent or reduced. For example, transgenic expression of the polynucleotide may lead to an abnormal state, e.g., a cancerous state, and transgenic animals expressing the polynucleotide may be useful models for disease.

In another embodiment, a polynucleotide of interest encodes a regulatory RNA molecule (e.g., an RNA molecule that is not substantially translated into a protein). Such regulatory RNA molecules include, but are not limited to, antisense RNA and RNA molecules that effect RNA interference (RNAi), e.g., siRNA (including shRNA) and microRNA (miRNA). RNAi generally involves the partial or complete silencing of genes by double-stranded RNA molecules, one strand of which is substantially or fully complementary to the coding region of a target gene. See Fire et al. (1998) Nature 391:806-811. For further review of RNAi, see; Novina and Sharp, Nature (2004) 430:161-164.

siRNAs have proven useful as a tool for modulating gene expression, e.g., where traditional antagonists such as small molecules or antibodies have failed or are otherwise not practicable. (Shi Y., Trends in Genetics 19(1):9-12 (2003)). In vitro synthesized, double stranded RNAs of 21 to 23 nucleotides in length have been shown to act as interfering RNAs (iRNAs) and can specifically inhibit gene expression (Fire A., Trends in Genetics 391; 806-810 (1999)). These iRNAs typically act by mediating degradation of their target mRNAs. Since iRNAs are generally (although not always) under 30 nucleotides in length, they generally do not trigger a cell antiviral defense mechanism, e.g., interferon production, and/or general shutdown of protein synthesis.

Practically, siRNAs can be synthesized and then cloned into nucleic acid constructs, such as those described herein. Such constructs can be introduced into mammalian cells, e.g., by microinjection or transfection, and/or can be used to create transgenic animals, e.g., as further described herein. siRNA may be expressed in a constitutive or inducible manner. siRNA may be expressed in a tissue specific manner, e.g., by operably linking the siRNA to a tissue-specific promoter. Expression of siRNA may be used to “knockdown” or significantly reduce the amount of protein encoded by the corresponding mRNA. Accordingly, siRNA may be useful to assess the phenotypic impact of knocking out the function of a gene of interest and/or to knock out a gene whose overexpression is believed to be linked to a disorder, e.g., cancer or inflammation. Thus, the present invention provides siRNA-based methods of modulating gene expression.

An siRNA may be expressed using any of the inducible expression systems described above. Suitable promoters for expression of siRNA include, but are not limited to, any of those described above, and in particular, pol III promoters, such as H1 or U6. Suitable siRNA for silencing a particular gene of interest are known in the art and/or may be routinely identified or designed by methods known to those skilled in the art. See, e.g., US 2005/0071893; Vickers et al. (2003) J. Biol. Chem. 278:7108-7118; Hill et al. (1999) Am. J. Respir. Cell Mol. Biol. 21:728-737; Sandy et al. (2005) Biotechniques 39:215-224.

d) Other Components

Other sequences may optionally be included in a nucleic acid construct of the invention. Such sequences include, but are not limited to, one or more enhancer sequences that are operably linked to a promoter(s) in a nucleic acid construct; one or more terminator sequences located 3′ of a polynucleotide to be transcribed from a nucleic acid construct; one or more IRES sequences; sequences that facilitate propagation of the construct; and/or cloning sites.

Many enhancer sequences from mammalian genes are known e.g., from globin, elastase, albumin, α-fetoprotein and insulin genes. A suitable enhancer is an enhancer from a eukaryotic cell virus. Examples include the SV40 enhancer on the late side of the replication origin (bp 100-270), the enhancer of the cytomegalovirus immediate early promoter (Boshart et al. Cell 41:521 (1985)), the polyoma enhancer on the late side of the replication origin, and adenovirus enhancers. See also Yaniv, Nature, 297:17-18 (1982) for discussion of enhancing elements for activation of eukaryotic promoters. Enhancer sequences may be 5′ or 3′ of a promoter. In certain embodiments, an enhancer is located at a site 5′ of the promoter or between the promoter and a polynucleotide to which it is operably linked.

A nucleic acid construct may optionally comprise an IRES. An IRES can be of varying length and from various sources, e.g., encephalomyocarditis virus (EMCV) or picornavirus genomes. Various IRES sequences and their construction are described in, e.g., Pelletier et al., Nature 334: 320-325 (1988); Jang et al., J. Virol. 63: 1651-1660 (1989); Davies et al., J. Virol. 66: 1924-1932 (1992); Adam et al. J. Virol. 65: 4985-4990 (1991); Morgan et al. Nucl. Acids Res. 20: 1293-1299 (1992); Sugimoto et al. Biotechnology 12: 694-698 (1994); and Ramesh et al. Nucl. Acids Res. 24: 2697-2700 (1996). In one embodiment, the IRES of ECMV is used in the nucleic acid constructs of the invention. A coding sequence operably linked to an IRES may be, for example, about 8 bases or more downstream of the 3′ end of the IRES or at any distance such that translation of the coding sequence occurs. The optimum or permissible distance between the IRES and the start of the downstream coding sequence can be readily determined by varying the distance and measuring expression as a function of the distance.

A nucleic acid construct may optionally comprise prokaryotic sequences that facilitate the propagation of the construct in bacteria. Therefore, the construct may comprise components such as an origin of replication (i.e., a nucleic acid sequence that enables the construct to replicate in one or more selected host cells) and antibiotic resistance genes for selection in bacteria. Origins of replication include, e.g., the ColE1 origin of replication in bacteria. Various viral origins (SV40, polyoma, adenovirus, VSV or BPV) are useful in mammalian cells where extrachromosomal (episomal) replication is desired. Additional eukaryotic selectable marker gene(s) may be incorporated.

A nucleic acid construct may comprise at least one cloning site for insertion or removal of a given sequence, for example, a polynucleotide whose expression is desired from an inducible promoter. In one embodiment, the cloning site is a multiple cloning site, i.e., containing multiple restriction sites. Gateway sites may also be used, permitting insertion of sequences using lambda-mediated recombination.

2. Specific Embodiments of Nucleic Acid Constructs

In addition to the above-provided embodiments, the following specific embodiments are further provided:

In one aspect, a nucleic acid construct is provided which comprises: (1) a first transcription unit comprising at least one polynucleotide of interest operably linked to an inducible promoter that comprises one or more TetO sequences; (2) a second transcription unit comprising a coding sequence encoding a TetR; and (3) a pair of inverted repeats, wherein one of the inverted repeats is 5′ of (1) and (2), and the other inverted repeat is 3′ of (1) and (2). In one embodiment, the TetR is expressed from the second transcription unit and represses the inducible promoter in the first transcription unit in the absence of an inducing agent. In the presence of an inducing agent (e.g., tetracycline or a tetracycline analog such as doxycycline), however, repression by TetR is relieved, and the polynucleotide of interest in the first transcription unit is thus expressed. In one such embodiment, the polynucleotide of interest encodes a regulatory RNA molecule capable of effecting RNAi (e.g., shRNA), such that expression of the nucleic acid sequence targeted by the regulatory RNA molecule is “knocked down” in the presence of the inducing agent. Thus, the nucleic acid constructs described herein provide a mechanism for regulated gene expression, and in particular, for conditional knock-down of target nucleic acid sequences.

In one embodiment, the first transcription unit is disposed 5′ of the second transcription unit. In another embodiment, the first transcription unit is disposed 3′ of the second transcription unit. In yet another embodiment, one or more additional transcription units (in addition to the first and second transcription units) may be present in a nucleic acid construct, as discussed further below, provided that such additional transcription units are contained within the inverted repeats.

a) Inverted Repeats

In one embodiment, the inverted repeats are selected from piggyBac, Sleeping Beauty, or Frog Prince, as described in further detail above. In one such embodiment, the inverted repeats are selected from piggyBac inverted repeats. In one such embodiment, at least one of the inverted repeats comprises a nucleic acid sequence selected from SEQ ID NOs:1, 2, 3, 4, or 5.

b) TetR

In one embodiment, the coding sequence encoding a TetR is optimized for expression in mammalian cells. In one such embodiment, the coding sequence is codon-optimized for expression in murine or human cells. In one such embodiment, the codon-optimized coding sequence comprises (a) the polynucleotide sequence below (with start and stop codons underlined):

(SEQ ID NO: 15)

ATGTCCAGACTGGATAAGTCCAAGGTGATTAATTCCGCTCTGGAACTCCT
GAACGAGGTCGGCATCGAGGGACTGACCACACGGAAGCTGGCTCAGAAAC
TCGGCGTCGAACAGCCTACCCTCTACTGGCATGTCAAAAATAAGAGAGCC
CTCCTGGACGCCCTGGCTATCGAGATGCTGGACAGACACCACACCCACTT
CTGCCCCCTGGAAGGCGAATCCTGGCAGGATTTCCTCCGGAACAACGCTA
AAAGCTTTAGATGCGCCCTCCTCAGCCATAGAGACGGAGCTAAAGTGCAC
CTGGGAACCCGGCCTACAGAAAAACAGTACGAGACACTGGAAAACCAGCT
CGCTTTCCTCTGCCAACAAGGCTTTAGCCTGGAAAACGCCCTCTACGCTC
TCAGCGCTGTCGGCCATTTTACACTGGGCTGCGTGCTCGAGGACCAGGAG
CACCAAGTGGCTAAAGAGAGCGGGAAACCCCTACCACCGATAGCATGCCC
CCCCTGCTGAGACAAGCCATTGAGCTCTTTGATCATCAGGGAGCTGAACC
CGCCTTCCTCTTTGGACTCGAACTCATTATTTGCGGACTCGAGAAGCAAC
TGAAATGCGAAAGCGGAAGCGCCTACTCCGGCTCCAGAGAATTTCGGTCC
TACTAG;
or

(b) a variant of SEQ ID NO:15 that encodes the same polypeptide as SEQ ID NO:15 and that is capable of being expressed in a mammalian cell at substantially similar levels as SEQ ID NO:15. In another embodiment, the amino acid sequence of a TetR encoded by nucleotides 1-507 of SEQ ID NO:15 is expressly provided. In another embodiment, the amino acid sequence of a TetR is shown in SEQ ID NO:21.

c) Inducible Promoter

In one embodiment, an inducible promoter comprises one or more TetO sequences. In one embodiment, an inducible promoter comprises a pol III promoter operably linked to one or more TetO sequences. In one such embodiment, an inducible promoter comprises an H1 promoter or a U6 promoter operably linked to one or more TetO sequences. In one embodiment, an inducible promoter comprises an H1 promoter operably linked to at least two TetO sequences. Such a promoter has been shown to be useful in embryonic stem cells and embryoid body cells, in which TetR-mediated repression was particularly stringent when a promoter comprising an H1 promoter operably linked to two TetO sequences was used. See co-pending U.S. application Ser. No. 11/460,606, filed Jul. 27, 2006, which is expressly incorporated by reference herein in its entirety. In one embodiment, an inducible promoter comprising two TetO sequences comprises: (a) the polynucleotide sequence of a “H1-tetO2-2X promoter segment” (from 5′ to 3′, with TetO sequences underlined):

(SEQ ID NO: 16)

CGAACGCTGACGTCATCAACCCGCTCCAAGGAATCGCGGGCCCAGT
GTCACTAGGCGGGAACACCCAGCGCGCGTGCGCCCTGGCAGGAAG
ATGGCTGTGAGGGACAGGGGAGTGGCGCCCTGCAATATTTGCATGT
CGCTATGTGTTCTGGGAAATCACCATAAACGTGAAATCCCTATCAG
TGATAGAGACTTATAAGTTCCCTATCAGTGATAGAGATCCCC;

(b) a polynucleotide comprising a polynucleotide that hybridizes under stringent conditions to the complement of the polynucleotide of (a); or (c) a polynucleotide comprising a polynucleotide that is at least about 90%, 95%, 96%, 97%, 98%, or 99% identical to the polynucleotide of (a), wherein the polynucleotide of (a), (b) or (c) is capable of being bound by TetR.

In one embodiment in which a pol III promoter is used, a pol III terminator sequence is disposed 3′ of the polynucleotide of interest. In one embodiment, a pol III terminator sequence comprises 4 or more consecutive T residues. In one such embodiment, a pol III terminator sequence comprises 5 consecutive T residues. In such embodiments, it is expected that pol III transcription stops at the second or third T, and accordingly, only 2 to 3 U residues will be added to the 3′ end of the RNA that is synthesized.

In one embodiment in which a pol II promoter is used, the polynucleotide of interest encodes an mRNA that encodes a polypeptide. In one embodiment in which a pol III promoter is used, the polynucleotide of interest encodes a regulatory RNA.

d) Transcription Units

In one embodiment, a transcription unit comprises a first coding region encoding a first RNA, and a second coding region encoding a second RNA, wherein both coding regions are under control of a common promoter (e.g., a pol III promoter). In one such embodiment, the first RNA and second RNA comprise sequences that are substantially or fully complementary and are therefore capable of forming an RNA molecule having a double-stranded region. Such double-stranded region may then function as an siRNA. For example, the first and second RNAs may comprise sequences of at least 10-30 contiguous nucleotides (including all integers between that range) that are complementary.

In one embodiment, a nucleic acid construct comprises multiple transcription units that encode one or more components of a regulatory RNA. For example, in one embodiment, a nucleic acid construct comprises (1) a first transcription unit comprising a first polynucleotide operably linked to a first promoter (e.g., a pol III promoter), wherein the first polynucleotide encodes a first RNA; and (2) a further transcription unit comprising a second polynucleotide operably linked to a second promoter (e.g., a second pol III promoter), wherein the second polynucleotide encodes a second RNA. In one such embodiment, the second RNA is substantially or fully complementary to the first RNA, such that the two RNAs can form a double-stranded structure when expressed. For example, in one such embodiment, the first and second RNAs may comprise sequences of at least 10-30 contiguous nucleotides (including all integers between that range) that are complementary.

In various embodiments, a nucleic acid construct comprises multiple transcription units encoding multiple regulatory RNAs that target different target nucleic acid sequences. In such embodiments, regulation of multiple endogenous genes may be achieved.

In another embodiment, a nucleic acid construct comprises a first promoter (e.g., a pol III promoter) operably linked to a polynucleotide that encodes an RNA, and a second promoter operably linked to the same polynucleotide but in the opposite orientation, such that expression of the polynucleotide from the first promoter results in synthesis of a first RNA, and expression of the polynucleotide from the second promoter results in synthesis of a second RNA that is substantially or fully complementary to the first RNA. For example, the first and second RNAs may comprise sequences of at least 10-30 contiguous nucleotides (including all integers between that range) that are complementary.

In one embodiment, the nucleic acid construct further comprises a selectable marker. In one such embodiment, the second transcription unit further comprises the selectable marker. In one such embodiment, an IRES (internal ribosome entry site) is disposed between the coding sequence encoding a TetR and the selectable marker.

e) Polynucleotide of Interest

In one embodiment, a polynucleotide of interest encodes an RNA (i.e., an mRNA) that is translated. In another embodiment, a polynucleotide of interest encodes a regulatory RNA molecule, e.g., one or both strands of a double-stranded RNA molecule. In one such embodiment, a regulatory RNA molecule forms a hairpin structure having a double-stranded region, e.g., an shRNA or a pre-miRNA.

In another embodiment, a regulatory RNA molecule comprises a double-stranded region, wherein one strand of the double-stranded region is substantially identical (typically at least about 80%, 85%, 90%, 91%, 92%, 93%, 94%, 95%, 96%, 97%, 98%, 99% identical) in sequence to a target nucleic acid sequence (e.g., a region of an mRNA derived from a gene of interest to be down regulated). The other strand of the double-stranded region is fully or partially complementary to the target nucleic acid (typically at least about 80%, 85%, 90%, 91%, 92%, 93%, 94%, 95%, 96%, 97%, 98%, 99% identical to the complement of a region of the target nucleic acid). It is understood that the double-stranded region can be formed by two separate RNA strands, or by self-complementary portions of a single RNA having a hairpin structure. The double-stranded region of a regulatory RNA molecule is generally at least about 10 nucleotides in length, at least about 15 nucleotides in length and, in some embodiments, is about 15 to about 30 nucleotides in length. However, a significantly longer double-stranded region can be used effectively. In one embodiment, the double-stranded region is between about 19 and 22 nucleotides in length (including any integer within that range). In one embodiment, one strand of the double-stranded region is identical to the target nucleic acid sequence over this region.

In another embodiment, a polynucleotide of interest encodes a regulatory RNA molecule that is self-complementary, such that the RNA molecule is capable of forming a hairpin structure comprising a “sense” region, a loop region and an “antisense” region. In one such embodiment, the sense and antisense regions are each about 15 to about 30 nucleotides in length. In another such embodiment, the loop region is from about 2 to about 15 nucleotides in length, or from about 4 to about 9 nucleotides in length. Following expression of such a regulatory RNA molecule, the sense and antisense regions form a double-stranded structure.

If a target nucleic acid sequence is derived from a gene that is a member of a highly conserved gene family, the sequence of the double-stranded region of a regulatory RNA molecule can be chosen with the aid of sequence comparison tools such that only the desired gene is down regulated. Alternatively, the sequence of a double-stranded region of a regulatory RNA molecule can be designed so that it will down regulate a plurality of related genes simultaneously.

Any of the above embodiments either singly or in combination with one another are expressly provided herein.

3. Cells and Animals Comprising Nucleic Acid Constructs

In one embodiment, a cell comprising any of the above-described nucleic acid constructs is provided. In one embodiment, a cell is a primary cell or a cultured cell from a cell line such as HEK, CHO, COS, MEF, and 293 cells. In another embodiment, a cell is a bacterial host cell. In another embodiment, a cell is a mammalian cell, such as a murine or human cell. Such a cell may be, e.g., an isolated cell (including a normal or diseased (e.g., cancerous) cell or a cell from a cell line); or an embryonic cell, such as an isolated embryonic cell, an embryonic stem (ES) cell, a single cell embryo (i.e., a fertilized egg), or a cell within an isolated embryo.

B. Methods

Methods are provided for introducing any of the above nucleic acid constructs into cells, e.g., mammalian cells. A nucleic acid construct may be introduced into a cell, e.g., by routine transfection methods or by microinjection, to create a transgenic cell. In one embodiment, a nucleic acid construct is introduced (e.g., transfected or microinjected) into an embryonic cell, such as a single cell embryo (i.e., a fertilized egg), a cell within an isolated embryo, or an embryonic stem (ES) cell. A transgenic embryonic stem cell may be combined with an embryo (e.g., a blastocyst-stage embryo). An embryo comprising any of the foregoing transgenic cells may be transferred to a pseudopregnant female animal (e.g., a mouse) to generate a transgenic animal. Alternatively, a transgenic embryonic stem cell may be cultured to form embryoid bodies comprising aggregates of differentiated cells.

In certain embodiments, a polynucleotide encoding a transposase contained in a separate vector is co-transfected or co-injected with a nucleic acid construct described herein. In certain embodiments, a polynucleotide encoding a transposase is contained in a nucleic acid construct provided herein.

Methods of using any of the above nucleic acid constructs are provided herein. For example, in one aspect, a method of expressing a polynucleotide of interest is provided, the method comprising introducing into a cell (a) any of the above nucleic acid constructs and (b) exposing the cell to a suitable inducing agent. In another embodiment, a method of inhibiting expression of an endogenous gene is provided, the method comprising introducing into a cell (a) any of the above nucleic acid constructs that inducibly expresses a regulatory RNA molecule (e.g., an shRNA) specific for the endogenous gene, and (b) exposing the cell to a suitable inducing agent. In any of the above methods, a cell may be present in a transgenic animal. In any of the above methods, a suitable transposase or a nucleic acid encoding such transposase is further introduced into the cell.

In another aspect, a method of expressing a polynucleotide of interest in a transgenic mammal is provided, the method comprising (a) introducing into a mammalian embryonic cell any of the above nucleic acid constructs; (b) introducing a suitable transposase or a nucleic acid encoding such transposase into the mammalian embryonic cell; (c) generating a transgenic mammal from the mammalian embryonic cell resulting from (a) and (b); and (d) administering to the transgenic mammal a suitable inducing agent. In one embodiment in which the method is used to inhibit expression of an endogenous gene in the transgenic mammal, the polynucleotide of interest encodes a regulatory RNA molecule (e.g., an shRNA) specific for the endogenous gene

In another aspect, a method of expressing a polynucleotide in a cell is provided, the method comprising (a) introducing into the cell a nucleic acid construct comprising: (i) a first transcription unit comprising the polynucleotide operably linked to an inducible promoter, wherein the inducible promoter comprises one or more TetO sequences; (ii) a second transcription unit comprising a coding sequence encoding a TetR; and (iii) a pair of inverted repeats, wherein one of the inverted repeats is 5′ of (i) and (ii), and the other of the inverted repeats is 3′ of (i) and (ii); and (b) exposing the cell to an inducing agent that induces expression of the polynucleotide from the inducible promoter.

In another aspect, a method of inhibiting expression of an endogenous gene in a cell is provided, the method comprising: (a) introducing into the cell a nucleic acid construct comprising: (i) a first transcription unit comprising a polynucleotide operably linked to an inducible promoter, wherein the inducible promoter comprises one or more TetO sequences, and wherein the polynucleotide encodes an shRNA specific for the endogenous gene; (ii) a second transcription unit comprising a coding sequence encoding a TetR; and (iii) a pair of piggyBac inverted repeats, wherein one of the inverted repeats is 5′ of (i) and (ii), and the other of the inverted repeats is 3′ of (i) and (ii); and (b) exposing the cell to an inducing agent that induces expression of the polynucleotide from the inducible promoter.

Therapeutic methods are also provided herein. In one aspect, a nucleic acid construct provided herein is used for in vivo gene therapy, e.g., to deliver a therapeutic nucleic acid to a target cell. Such in vivo gene therapy applications have been demonstrated using Sleeping Beauty transposon-based systems. See U.S. Pat. No. 6,613,752. A therapeutic nucleic acid may be a coding sequence that replaces the function of a defective endogenous gene in the target cell or that has utility in the treatment of a disease such as cancer or an immune disorder.

Therapeutic nucleic acids for use in the treatment of genetic defect-based disease conditions include, but are not limited to, coding sequences encoding the following: factor VIII, factor IX, beta-globin, low-density protein receptor, adenosine deaminase, purine nucleoside phosphorylase, sphingomyelinase, glucocerebrosidase, cystic fibrosis transmembrane regulator, alpha-antitrypsin, CD-18, ornithine transcarbamylase, arginosuccinate synthetase, phenylalanine hydroxylase, branched-chain alpha.-ketoacid dehydrogenase, fumarylacetoacetate hydrolase, glucose 6-phosphatase, alpha-L-fucosidase, beta-glucuronidase, alpha-L-iduronidase, galactose 1-phosphate uridyltransferase, and the like.

Therapeutic nucleic acids for the treatment of cancer include, but are not limited to, the following: coding sequences that encode tumor suppressors, toxins, suicide proteins, and the like; and polynucleotides that encode regulatory RNA for inhibiting expression of endogenous genes, e.g., regulatory RNA specific for cancer promoting genes. Such cancer promoting genes include, but are not limited to, oncogenes, such as ABLI, BCLI, BCL2, BCL6, CBFA2, CBL, CSFIR, ERBA, ERBB, ERBB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES; genes that promote angiogenesis, such as VEGF, VEGF receptor, and erythropoietin; and other cancer promoting genes such as PTI-1, PTI-2, and PTI-3. Therapeutic nucleic acids for the treatment of immune disorders include, but are not limited to, polynucleotides that encode regulatory RNA for inhibiting expression of endogenous genes, e.g., regulatory RNA specific for genes involved in inflammation, including, but not limited to, cytokines and chemokines.

Various methods may be used to introduce nucleic acids into cells. The techniques vary depending upon whether the nucleic acid is transferred into cultured cells in vitro, ex vivo or in vivo. For example, methods for introducing nucleic acid into a patient's cells include in vivo and ex vivo methods. In ex vivo methods, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). In vivo nucleic acid transfer techniques include lipid-based systems (useful lipids for lipid-mediated transfer of nucleic acids are DOTMA, DOPE and DC-Chol, for example). Nucleic acids contained within transposon-based vectors may be administered directly (e.g., intravenously) into a patient. See U.S. Pat. No. 6,613,752. For review of currently known gene marking and gene therapy protocols see Anderson et al., Science 256:808-813 (1992). See also WO 93/25673 and the references cited therein. Techniques suitable for the transfer of nucleic acid into mammalian cells in vitro include the use of liposomes, electroporation, microinjection, cell fusion, DEAE-dextran, the calcium phosphate precipitation method, etc.

It is understood that therapeutic agents discussed herein, including nucleic acid molecules, can be modified or synthesized to improved their bioavailability, pharmacokinetic and pharmacodynamic properties. For example, therapeutic nucleic acid molecules can be synthesized with one or more phosphorothioate linkages using techniques known in the art.

### III. EXAMPLES

### A. Construction of PiggyBac-Based Vector for Inducible Expression of shRNA

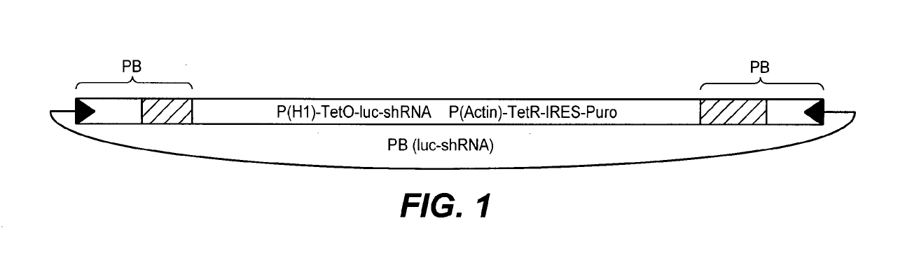

A piggyBac-based vector was constructed for inducible expression of shRNA specific for luciferase. The vector, referred to as “PB(luc-shRNA),” is shown in FIG. 1. PB(luc-shRNA) comprises piggyBac IRs flanking two internal transcription units in a pBluescript (Stratagene, La Jolla, Calif.) backbone. The first transcription unit comprises (from 5′ to 3′) the H1-TetO2-2x promoter (SEQ ID NO:16), referred to as “p(H1)-TetO” in FIG. 1, operably linked to a polynucleotide that encodes shRNA specific for the luciferase gene (referred to as “luc-shRNA” in FIG. 1). A polyadenylation signal follows the polynucleotide encoding shRNA. The second transcription unit comprises the human β-actin promoter and HTLV enhancer (referred to as “P(actin)” in FIG. 1) operably linked to a TetR-IRES-puromycin cassette. The TetR coding sequence (SEQ ID NO:15) is codon-optimized. The first and second transcription units are flanked by sequences comprising piggyBac IRs (referred to as “PB” in FIG. 1). One skilled in the art would understand that the above-described vector can be adapted for the expression of an shRNA specific to any target nucleic acid.

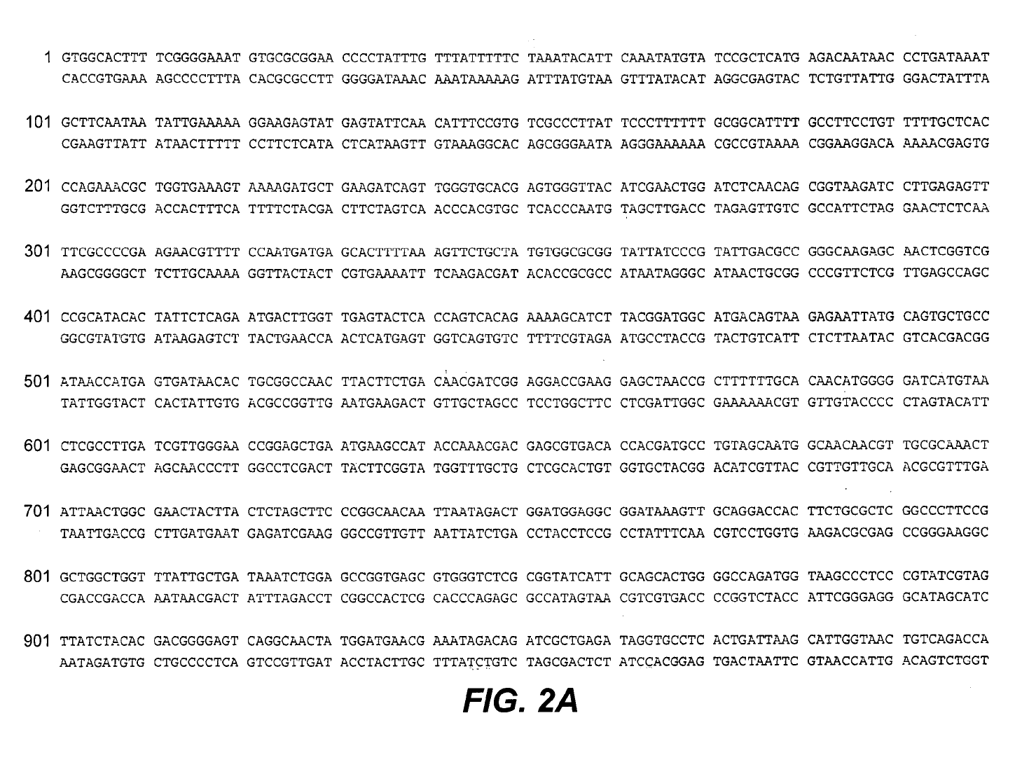

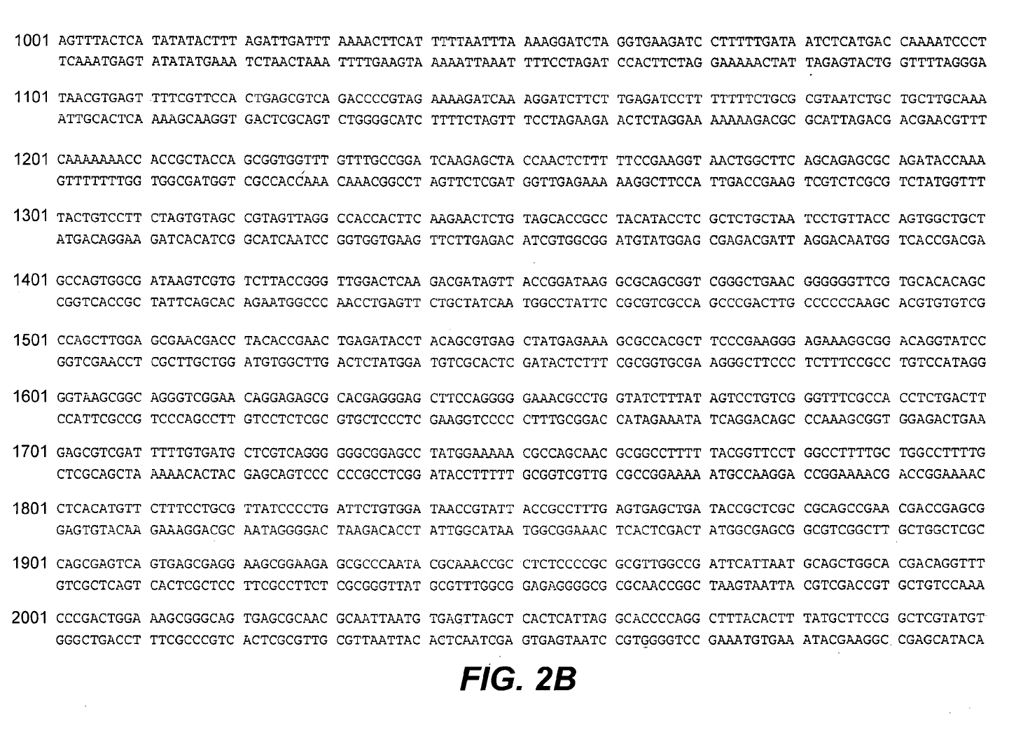

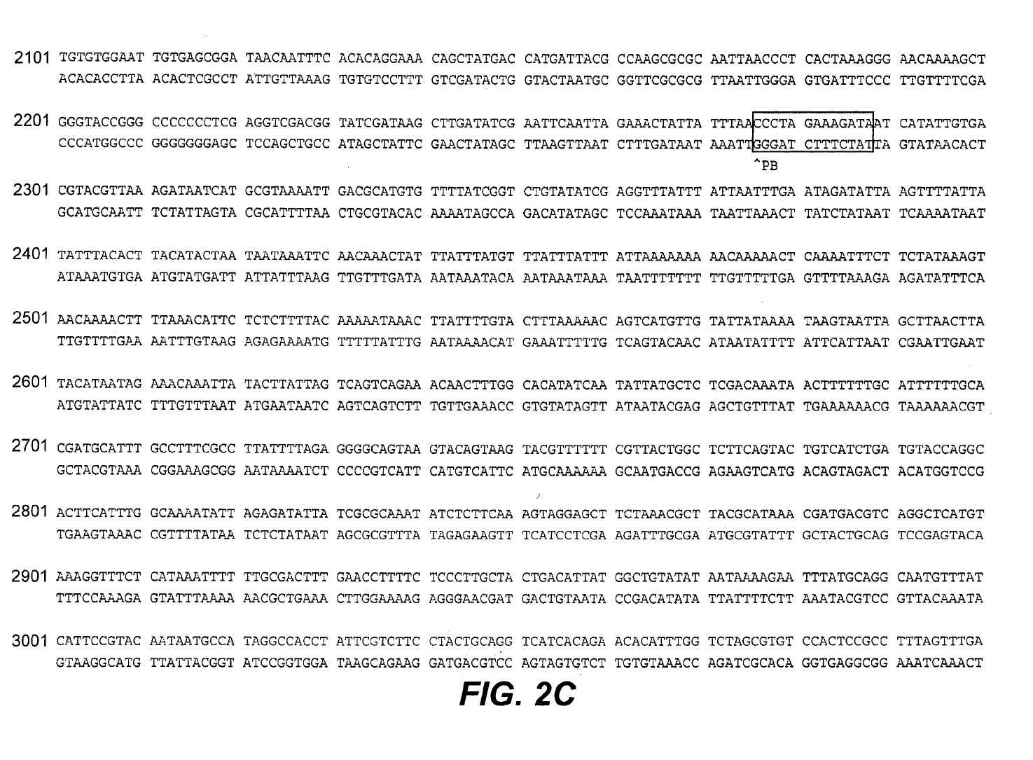

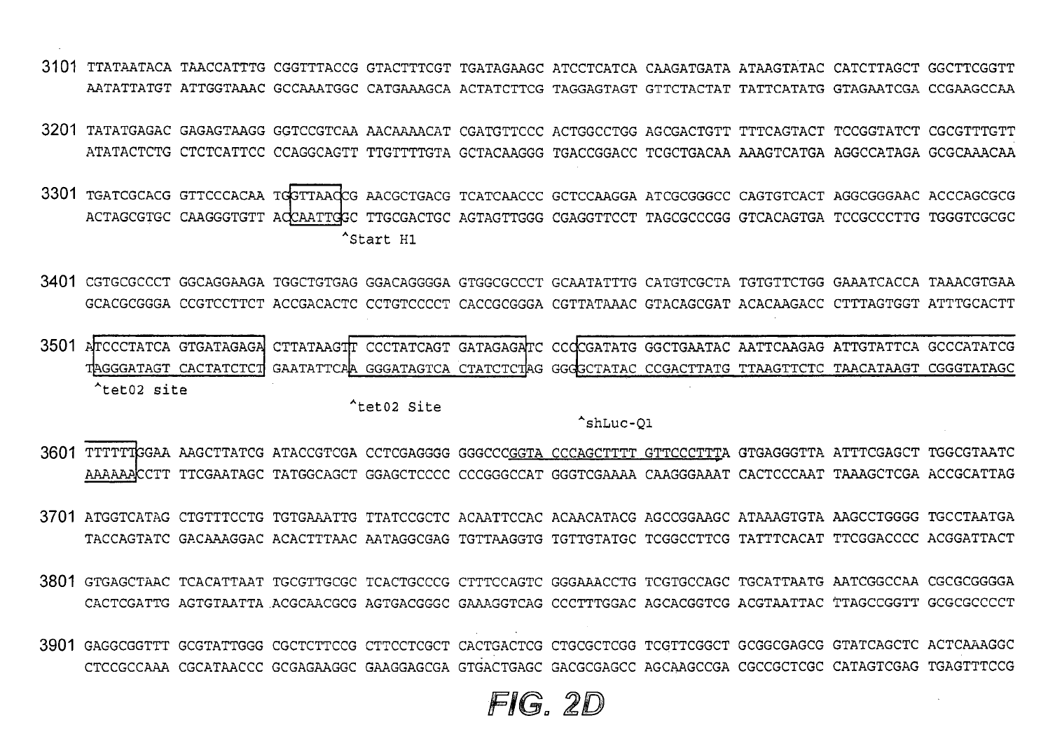

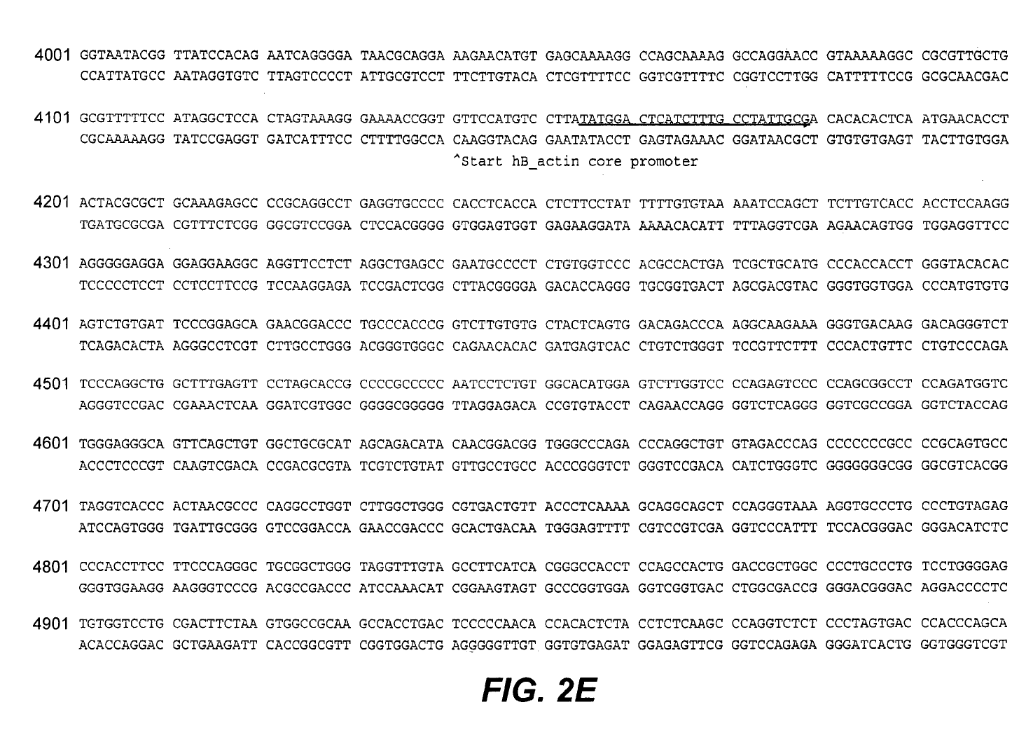

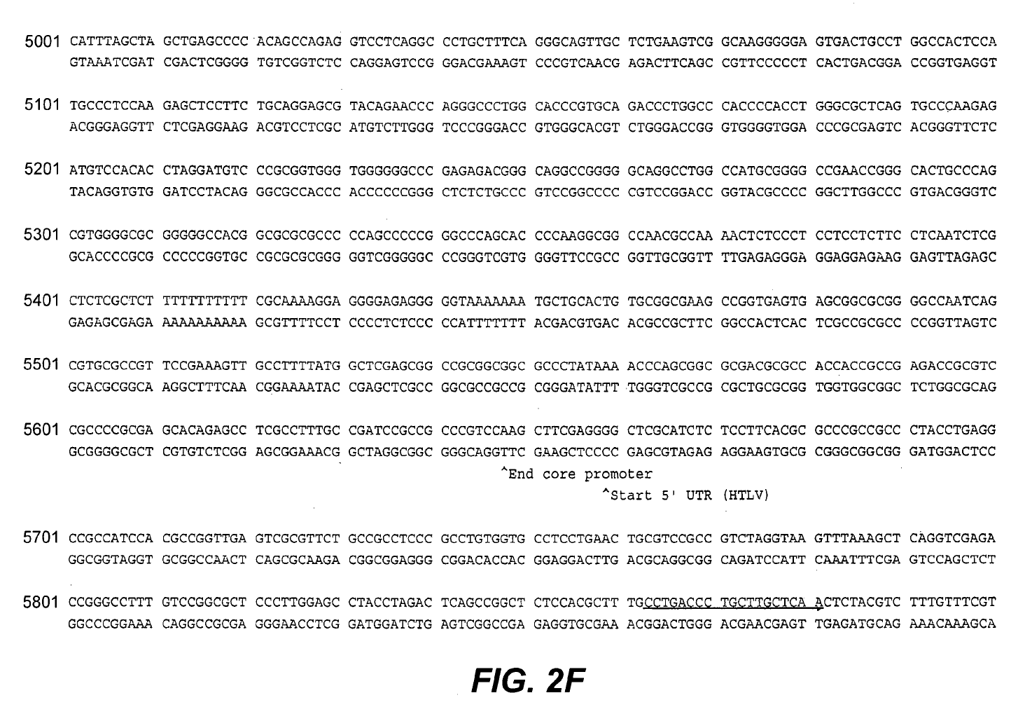

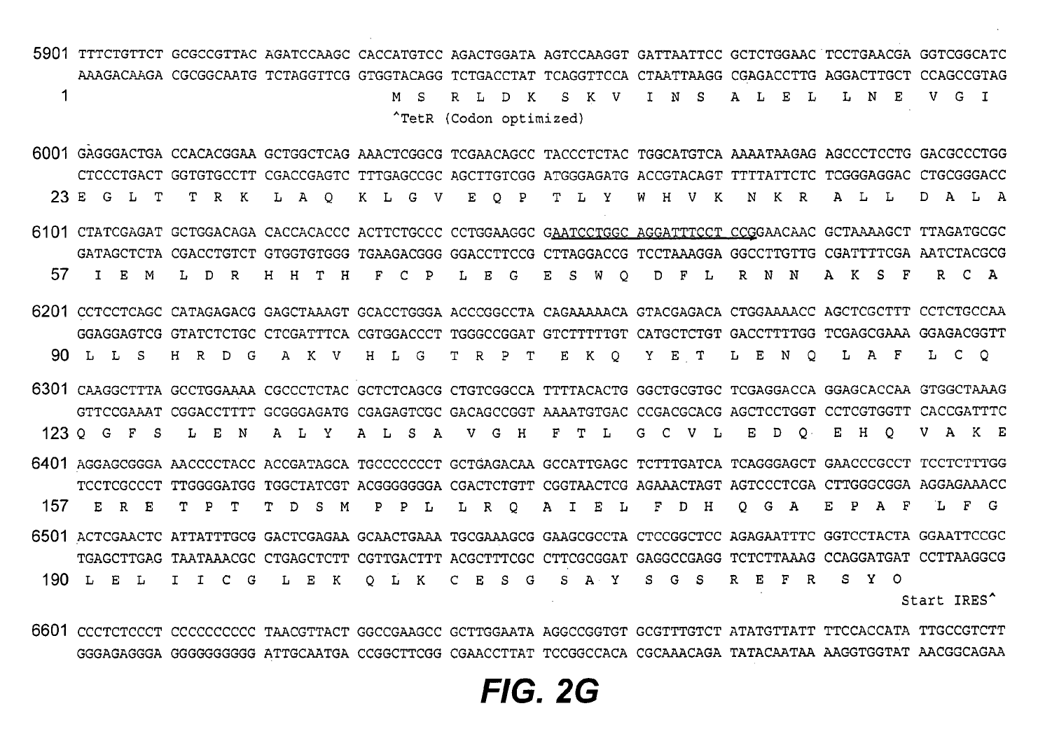

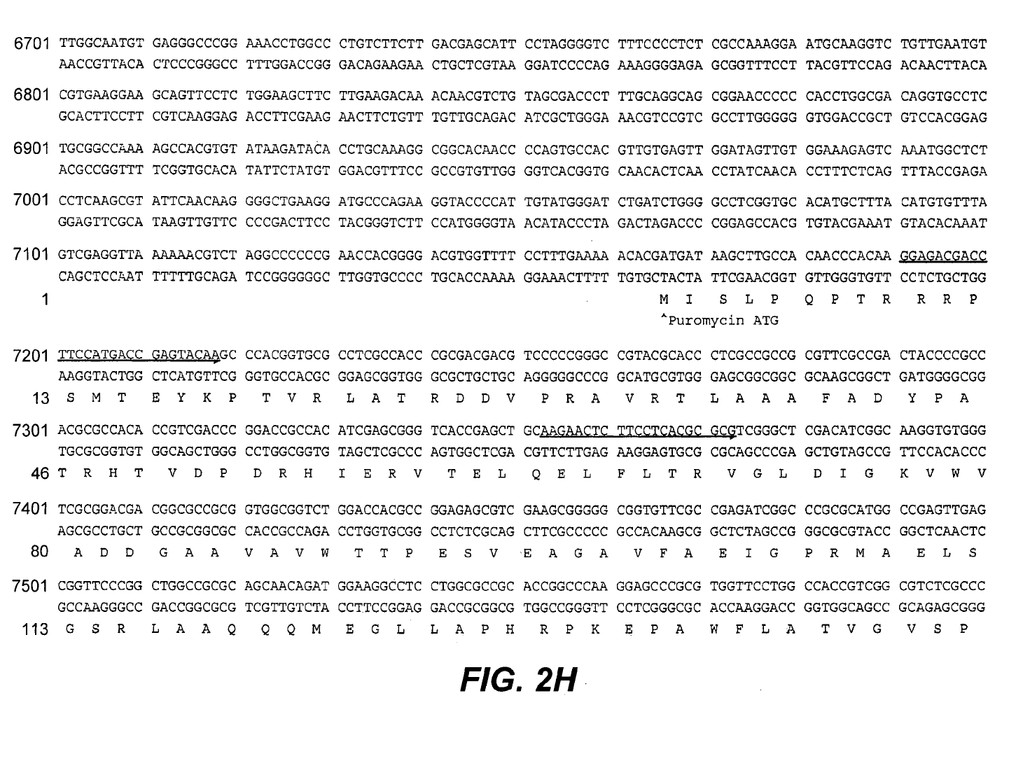

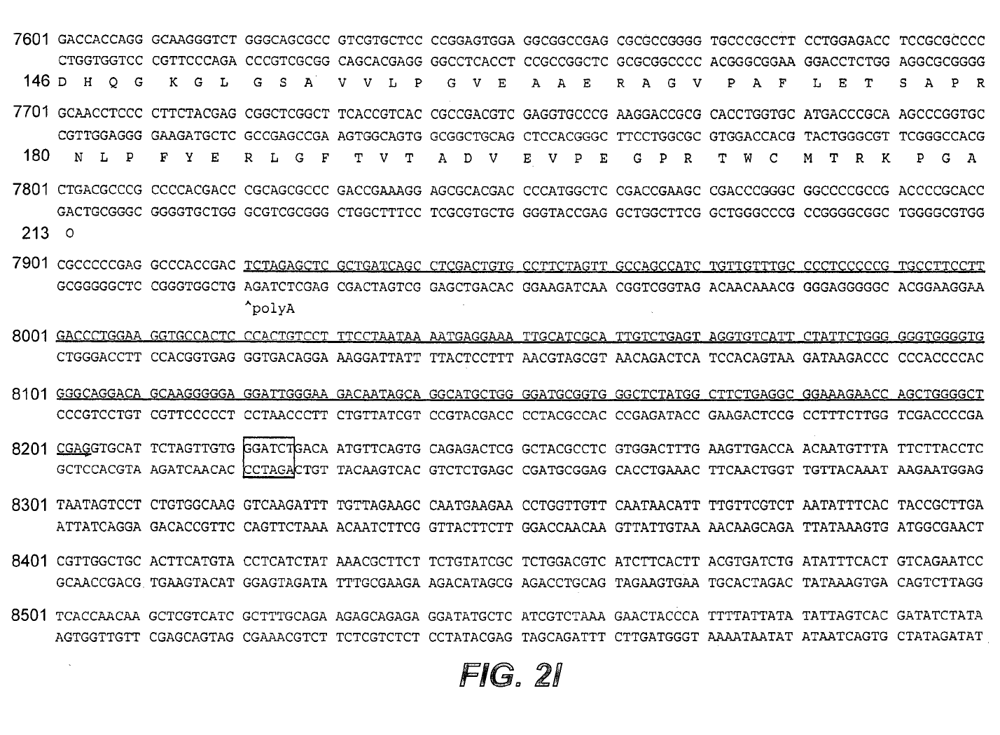

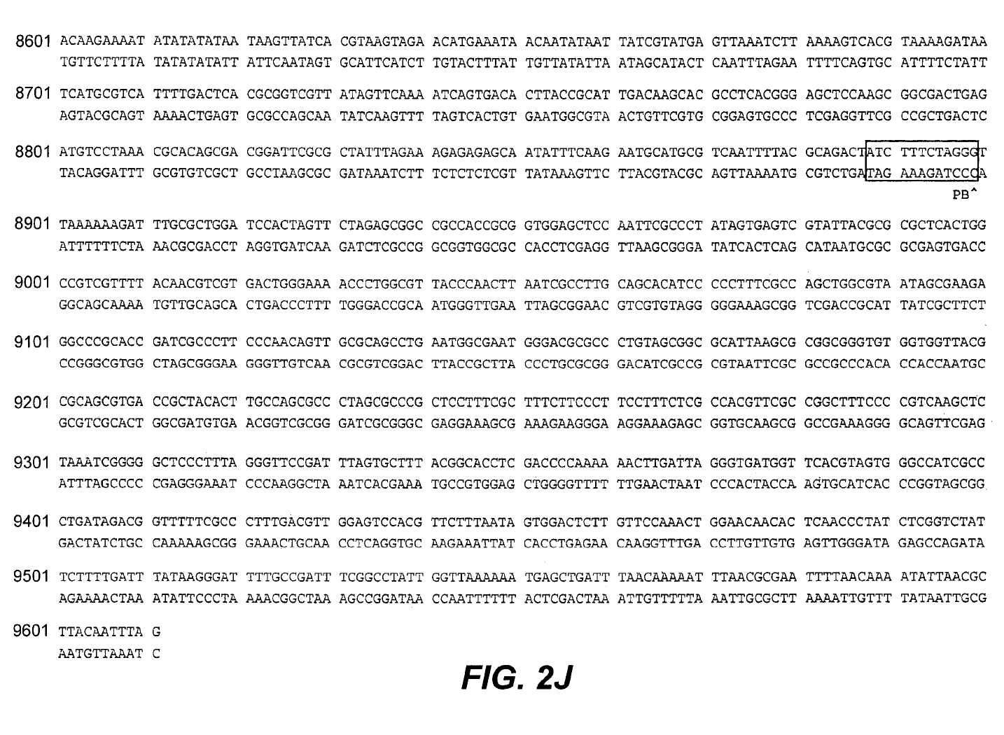

PB(luc-shRNA) was constructed as follows. A plasmid containing the second transcription unit was constructed using routine recombinant methods. PCR was used to generate an amplicon containing the first transcription unit, and the amplicon was subcloned into the plasmid upstream of the second transcription unit. PCR was then used to generate an amplicon containing the first and second transcription units. That amplicon was then subcloned between sequences comprising piggyBac IRs, which were contained within a pBluescript backbone (Stratagene, La Jolla, Calif.). The sequence of the entire PB(luc-shRNA) construct is shown in FIG. 2 and SEQ ID NO:17. The functional elements of the construct are also annotated in FIG. 2.

PB(luc-shRNA) functions as follows, and as exemplified in further detail below. In the ‘off’ state, the Tet repressor protein (TetR) is constitutively expressed and binds the TetO sequences in the H1-TetO2-2x promoter, thereby inhibiting shRNA expression. However, in the presence of the tetracycline analog, doxycycline (Dox), TetR protein is released from the promoter, permitting shRNA transcription. Thus, in the presence of Dox, luciferase expression is knocked down, and accordingly, bioluminescence is decreased.

### B. Doxycycline Induces Knock Down of Luciferase Gene Expression in ES Cells Transfected with PB(luc-shRNA)

To test whether PB(luc-shRNA) inducibly expresses shRNA specific for luciferase, ES cells and transgenic animals expressing luciferase were generated. A nucleic acid construct, referred to as “Rosa26-luciferase,” for high level expression of luciferase from the Rosa26 promoter was generated. The Rosa26 promoter directs expression of luciferase throughout all tissues in transgenic animals, thereby allowing whole body imaging. See PCT/US2006/039035, filed Oct. 10, 2006. The Rosa26-luciferase construct was made by cloning the murine Rosa26 promoter as a 1.9 Kb Hind III-Xba I fragment derived from pBROAD3 (InvivoGen, San Diego, Calif.) into a vector containing the 1.7 Kb luciferase gene using convenient restriction sites. A polyadenlyation site was added to the 3′ end of the luciferase gene for better expression of luciferase.

The Rosa26-luciferase construct was co-transfected into ES cells with a Neo resistance plasmid (10:1) and selected in G418. Luciferase positive cells (referred to as “Rosa-luc ES cells”) were chosen for further study. Those cells were transfected by electroporation with PB(luc-shRNA) and selected with puromycin. The selected cells were treated with either 0.5 μg/ml or 1 μg/ml doxycycline (Dox) for 7 days in the presence of 0.8 mg/ml luciferin (a luciferase substrate). As shown in FIG. 3, increasing concentrations of Dox resulted in decreasing bioluminescence (i.e., decreasing luciferase activity) relative to the control (“No Dox”) cells, indicating that Dox induced expression of shRNA specific for luciferase. Each row shows increasingly longer exposures of the imaged cells.

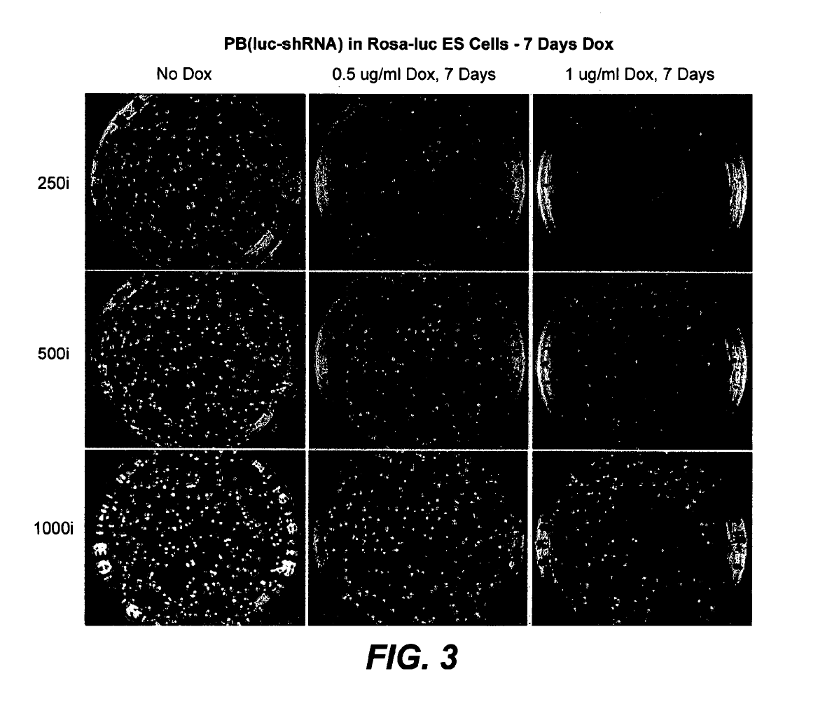

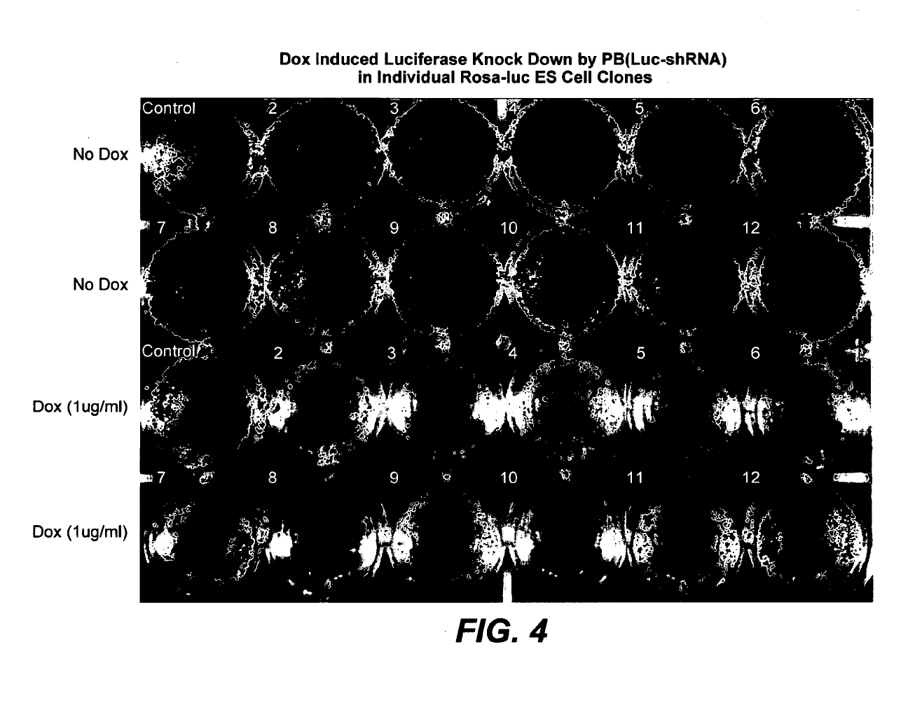

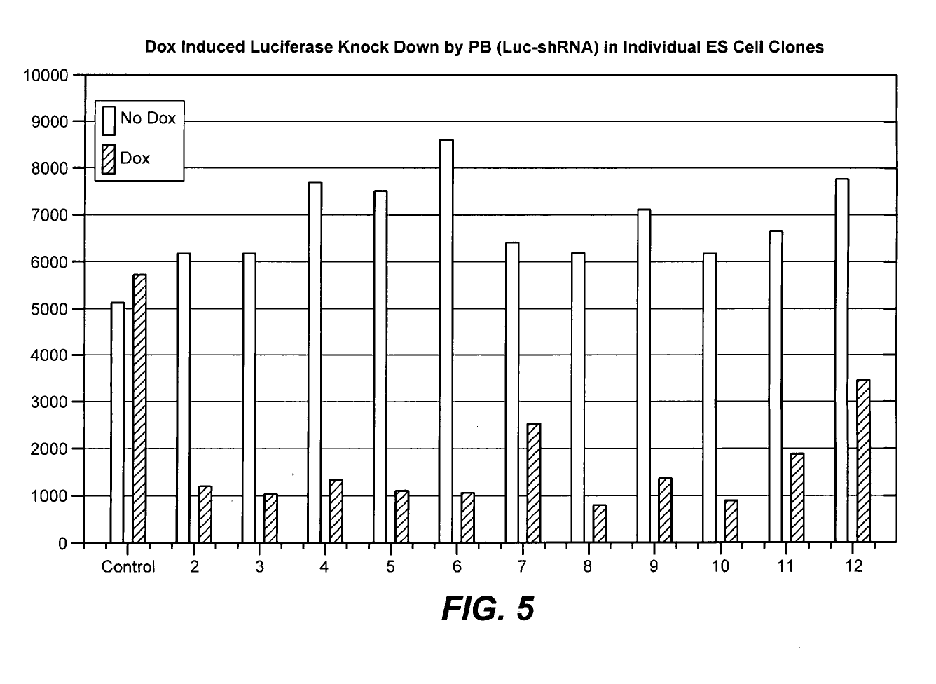

Rosa-luc ES cells transfected with PB(luc-shRNA) were individually cloned. The clones were treated with 1 μg/ml Dox for 3 days in the presence of luciferin. As shown in FIG. 4, Dox treated cells showed significantly decreased bioluminescence compared to the cells not treated with Dox. (Samples labeled with “Control” in FIG. 4 refer to clones that were not transfected with PB(luc-shRNA).) The reduction in bioluminescence was quantified for each individual Rosa-luc ES clonal cell line, as shown in FIG. 5. The y axis in FIG. 5 measures the counts of recorded signal in FIG. 4.

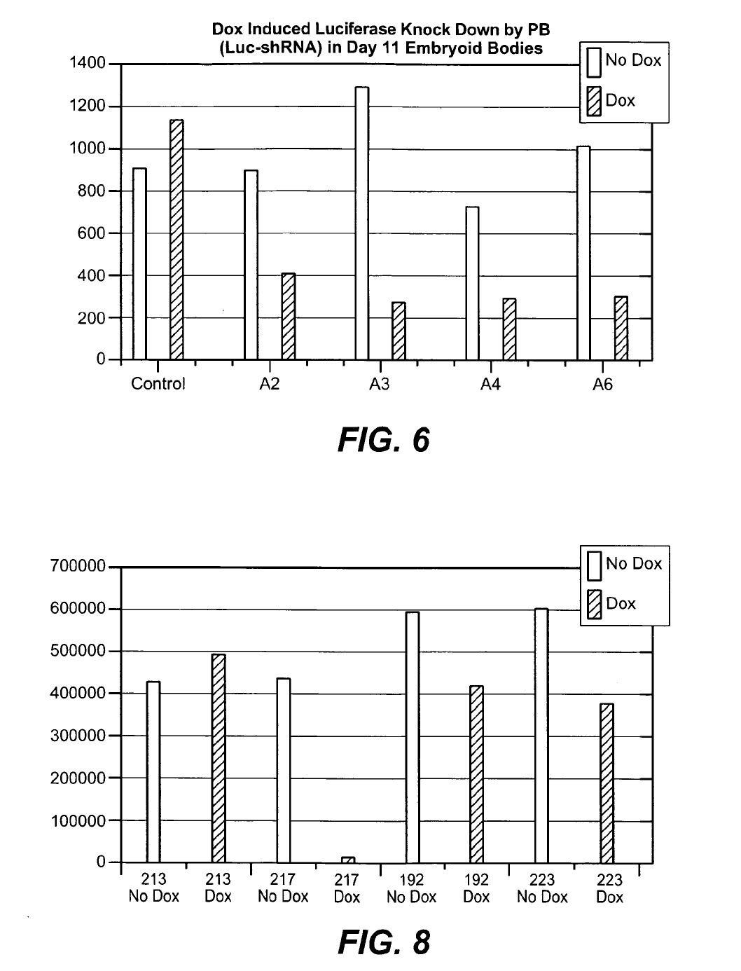

Rosa-luc ES cells transfected with PB(luc-shRNA) were induced to differentiate into embryoid bodies (EBs) for 11 days. EBs are a powerful tool to study gene function because they recapitulate early embryonic development in vitro. Differentiation was induced by culturing the cells in hanging drops (−600 cells/30 μl/drop) of differentiation medium (DMEM supplemented with 10% heat-inactivated fetal calf serum, 50 U/ml penicillin, and 50 μg/ml streptomycin, with or without 1.0 μg/ml doxycycline). The cells were then cultured in differentiation medium in suspension as EBs in bacteriological petri dishes for 7 days. EBs were then transferred to tissue culture plates coated with 0.1% gelatin for continued culture in differentiation medium for a total of 11 days. As shown in FIG. 6, EBs which were cultured in medium containing doxycycline showed significantly decreased bioluminescence compared with EBs cultured in medium that did not contain doxycycline. (Bars labeled with “Control” in FIG. 5 refer to clones that were not transfected with PB(luc-shRNA).)

### C. Doxycycline Induces Knock Down of Luciferase Gene Expression in PB(luc-shRNA) Transgenic Animals

The Rosa26-luciferase construct was injected into oocytes from FVB mice to generate transgenic “Rosa-luc” mice using routine methods described in PCT/US2006/039035, filed Oct. 10, 2006. Transgenic founders were analyzed and one line with ubiquitous and strong luciferase expression was used for the following experiment.

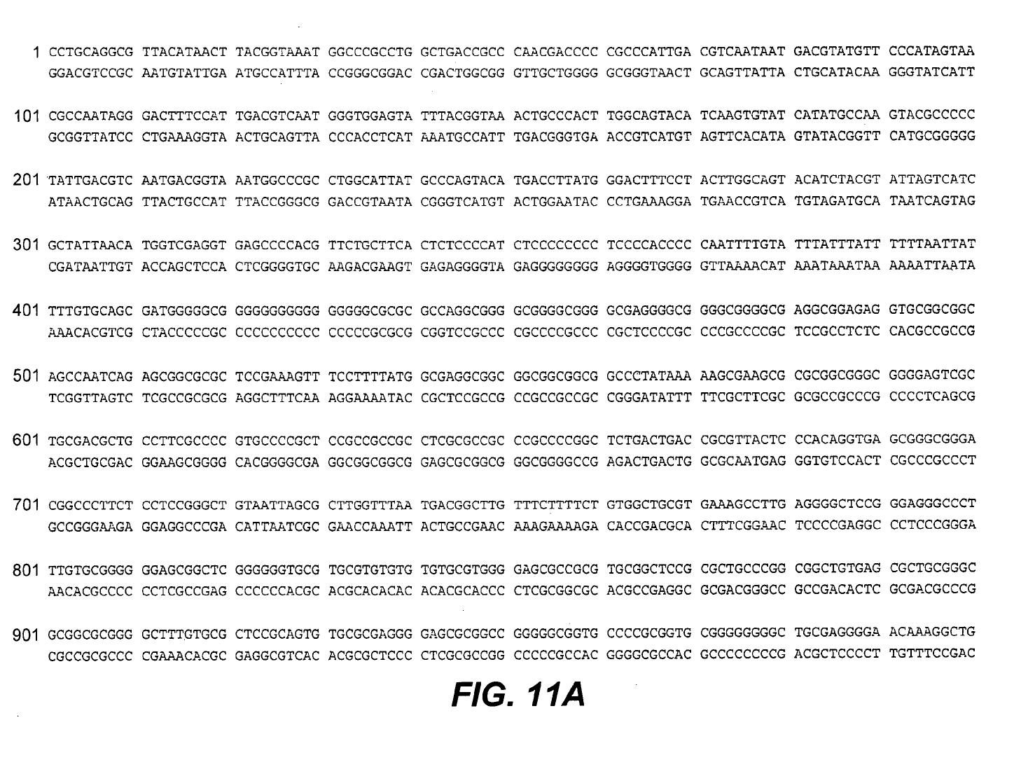

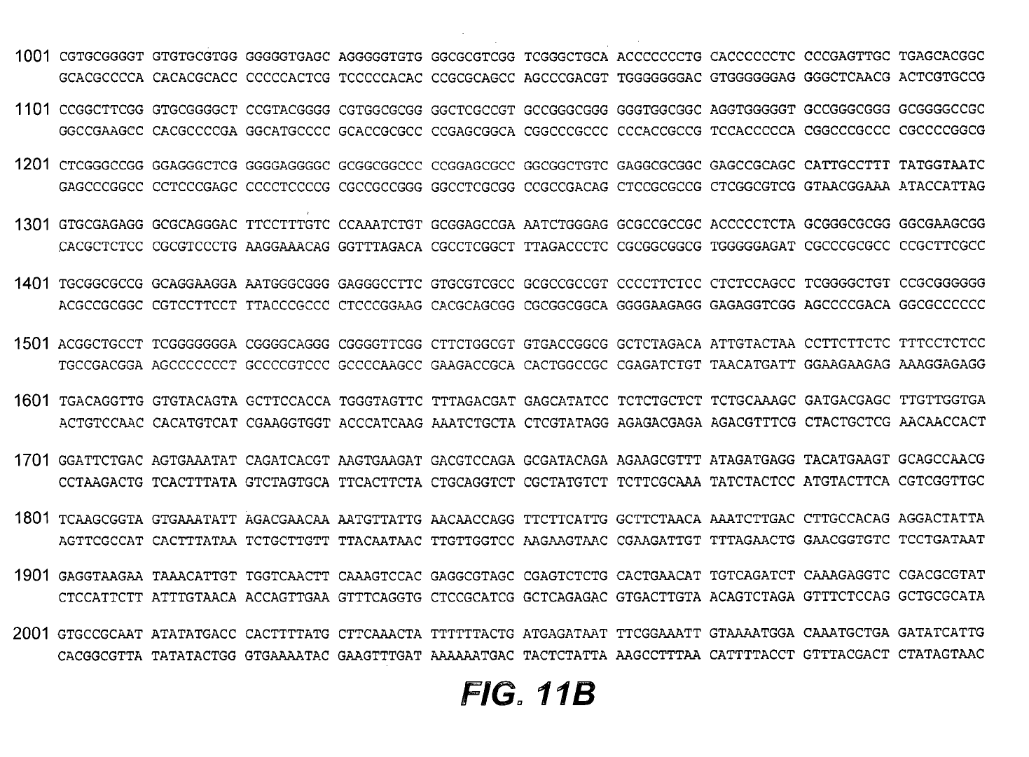

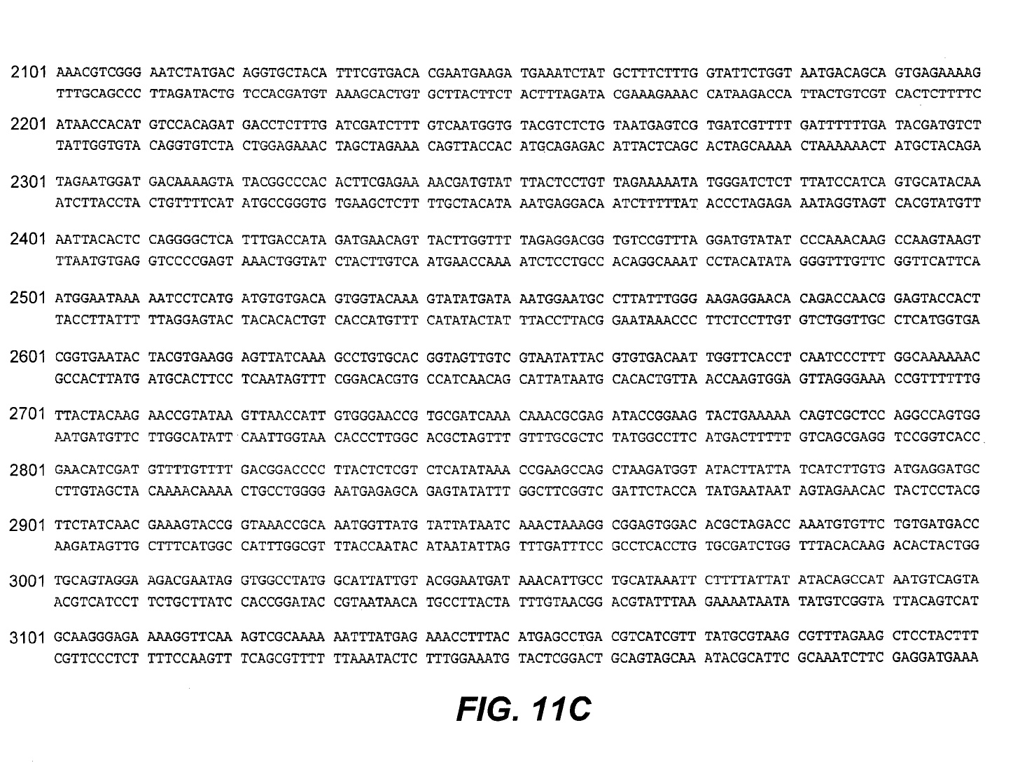

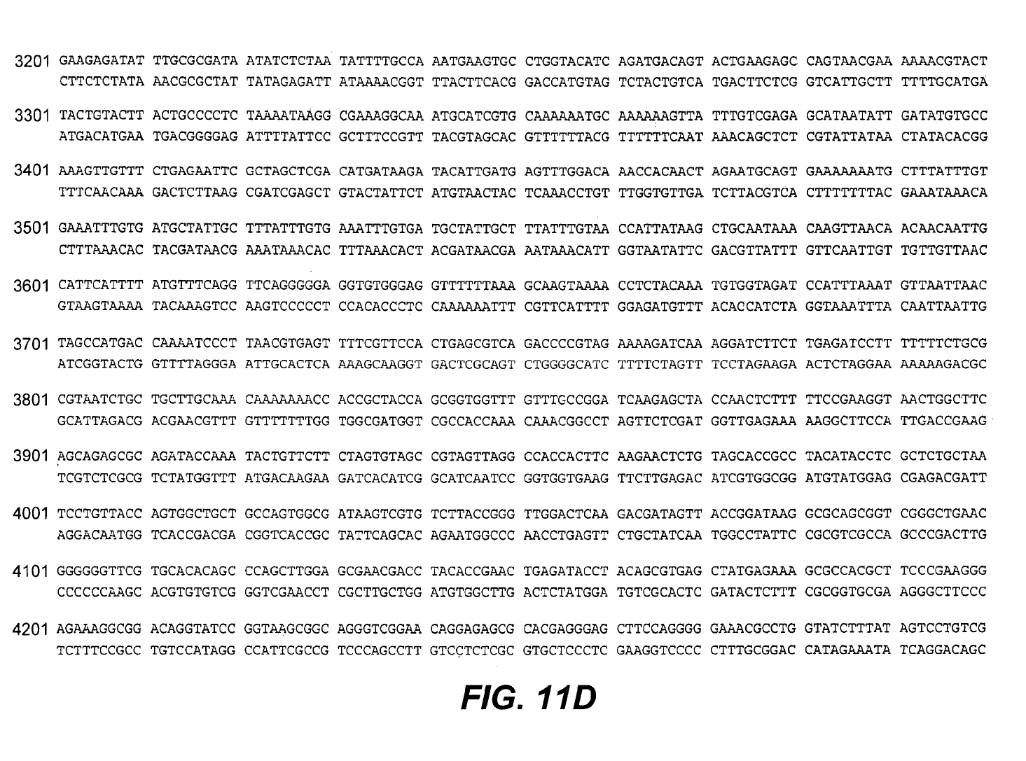

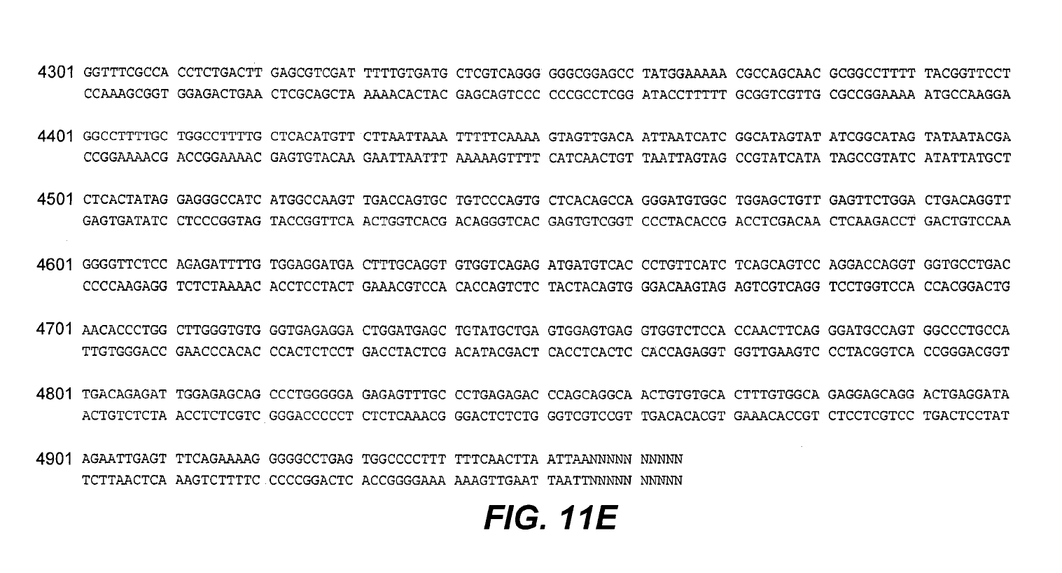

A vector was constructed in which nucleic acid encoding piggyBac transposase was placed under control of a promoter comprising the human β-actin promoter and CMV enhancer (InvivoGen, San Diego, Calif.). The nucleic acid sequence of the resulting vector (“pCAG-PBase”) is shown in FIG. 11 and SEQ ID NO:18. pCAG-PBase at a concentration of 1 ng/μl and PB(luc-shRNA) at a concentration of about 1-3.2 ng/μl were co-injected into pronuclei of single cell Rosa-luc mouse embryos (i.e., fertilized eggs). The injected embryos were transferred to surrogate female mice. Progeny were genotyped by PCR to identify stable germline transmission of PB(luc-shRNA). Those mice were bred to generate mouse lines referred to as “PB(luc-shRNA)/Rosa-luc” mouse lines.

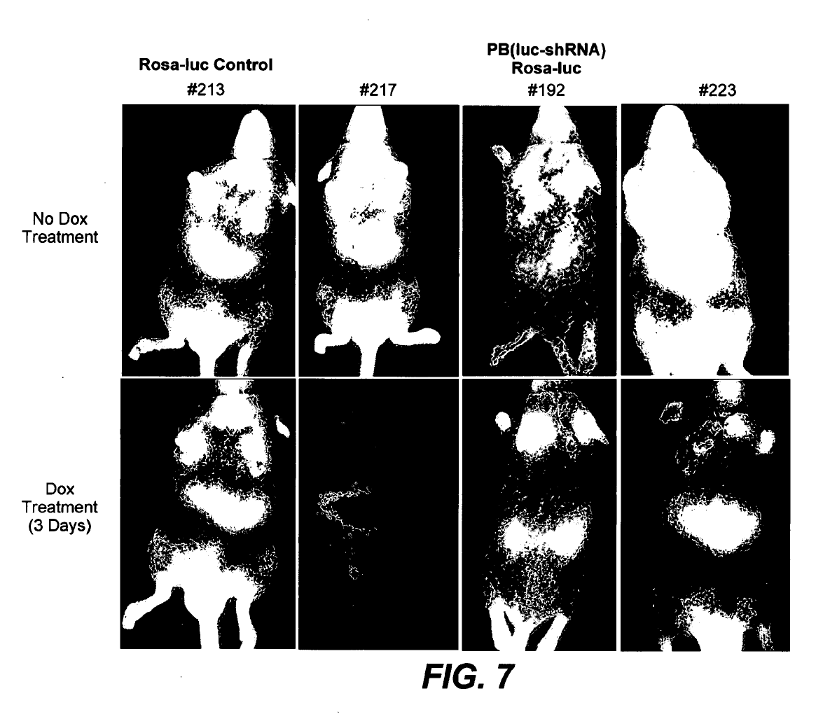

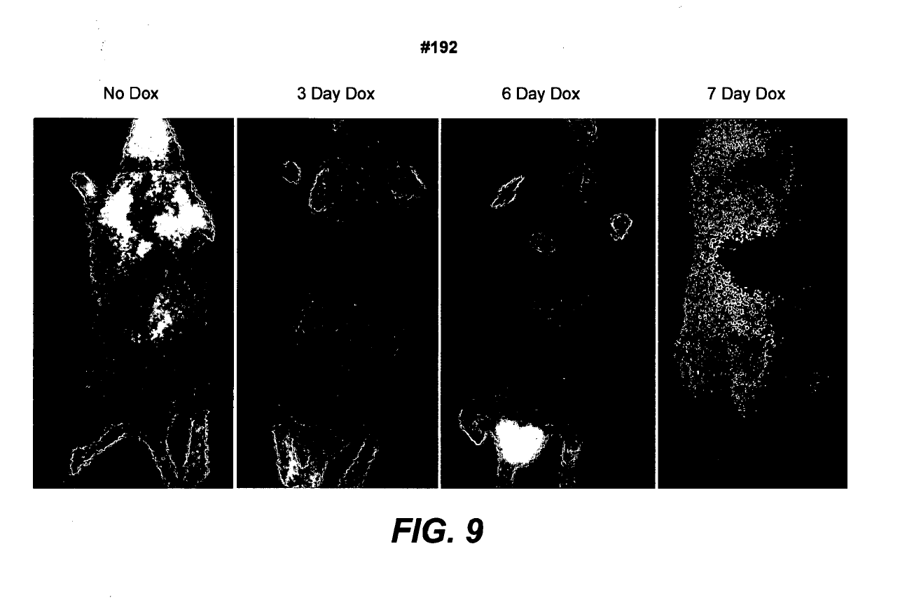

Doxycyline was administered to PB(luc-shRNA)/Rosa-luc mice by giving the mice drinking water containing 0.2 mg/ml of doxycycline and 0.5% sucrose for up to a week. The mice were then injected intraperitoneally with 250 μl of luciferin at 20 mg/ml. The mice were then anesthetized and subjected to whole body imaging using a CCD camera. The results are shown in FIG. 7. The Rosa-luc control mouse (mouse #213), which does not contain a PB(luc-shRNA) transgene, did not show decreased bioluminescence after treatment with doxycycline. One of the PB(luc-shRNA)/Rosa-luc mice (mouse #217) showed a dramatic decrease in bioluminescence after three days of induction, indicating that doxycycline induced expression of the luciferase-specific shRNA, which subsequently knocked down luciferase expression. The other two PB(luc-shRNA)/Rosa-luc mice (mouse #s192 and 223) showed about a 33% decrease in bioluminescence. The results shown in FIG. 7 are quantified in FIG. 8. To further explore the effect of doxycycline on PB(luc-shRNA)/Rosa-luc mice, we continued to treat mouse #192 with Dox for a total of 7 days. As shown in FIG. 9, the level of luciferase expression from mouse #192 was dramatically reduced by day 7. These results suggest that induction of shRNA specific for luciferase may depend upon the efficiency and/or duration of doxycycline delivery.

### D. Construction of PiggyBac-Based Vector for Inducible Expression of shRNA Specific for Lipin or Other Genes

A piggyBac-based vector for inducible expression of shRNA specific for lipin was constructed as described below:

An shRNA shuttle vector was constructed by amplifying the pSuperior H1 promoter (OligoEngine, Seattle, Wash.) by PCR, followed by TOPO-cloning of the amplification product into pENTR/D (Invitrogen, Carlsbad, Calif.). The following oligos were then ligated into the MslI and HindIII sites of the vector to generate pShuttle-H1:

Sense oligo

(SEQ ID NO: 19)

5′ ACGTGAAATCCCTATCAGTGATAGAGACTTATAAGTTCCCTATCAG
TGATAGAGATCTAAAGGGAAAA 3′
Anti-sense oligo

(SEQ ID NO: 20)

5′ AGCTTTTTCCCTTTAGATCTCTATCACTGATAGGGAACTTATAAGTC
TCTATCACTGATAGGGATTTCACGT 3″
(tetO underlined)

The resulting promoter (“H1-TetO2-2x”) in pShuttle-H1 comprised the H1 promoter and two TetO sequences, one of which was positioned between the TATA box and the transcriptional start site, and the other of which was positioned upstream of the TATA box. The polynucleotide sequence of the H1-TetO2-2x promoter in pShuttle-H1 comprises the following sequence:

(SEQ ID NO: 16)

CGAACGCTGACGTCATCAACCCGCTCCAAGGAATCGCGGGCCCAGTGTCA
CTAGGCGGGAACACCCAGCGCGCGTGCGCCCTGGCAGGAAGATGGCTGTG
AGGGACAGGGGAGTGGCGCCCTGCAATATTTGCATGTCGCTATGTGTTCT
GGGAAATCACCATAAACGTGAAATCCCTATCAGTGATAGAGACTTATAAG
TTCCCTATCAGTGATAGAGATCCCC.

In pShuttle-H1, the H1-TetO2-2x promoter is flanked by the Gateway recombination sites attL1 and attL2 so that the promoter (and any polynucleotide subcloned downstream of the promoter but upstream of attL2) may be easily transferred by Gateway recombination.

Three different BglII-HindIII fragments encoding shRNAs specific for the lipin gene were each ligated into pShuttle-H1 downstream of the TetO sequences. The three fragments were generated using the following three sets of oligonucleotides (underline indicates region of self-complementarity):

Set 1
Lipin-shRNA OL3A

(SEQ ID NO: 23)

GATCCCCCGACAACCCTGCTATCATCTTCAAGAGAGATGATAGCAGGGTT
GTCGTTTTTTGGAAA
Lipin-shRNA OL3B

(SEQ ID NO: 24)

AGCTTTTCCAAAAAACGACAACCCTGCTATCATCTCTCTTGAAGATGATA
GCAGGGTTGTCGGGG
Set 2
Lipin-shRNA OL5A

(SEQ ID NO: 25)

GATCCCCGGTTGACGCCAAAGAATAATTCAAGAGATTATTCTTTGGCGTC
AACCTTTTTTGGAAA
Lipin-shRNA OL5B

(SEQ ID NO: 26)

AGCTTTTCCAAAAAAGGTTGACGCCAAAGAATAATCTCTTGAATTATTCT
TTGGCGTCAACCGGG
Set 3
Lipin-shRNA OL8A

(SEQ ID NO: 27)

GATCCCCCCGGAAGACTCCTGATAAATTCAAGAGATTTATCAGGAGTCT
TCCGGTTTTTTGGAAA
Lipin-shRNA OL8B

(SEQ ID NO: 28)

AGCTTTTCCAAAAAACCGGAAGACTCCTGATAAATCTCTTGAATTTATCA
GGAGTCTTCCGGGGG

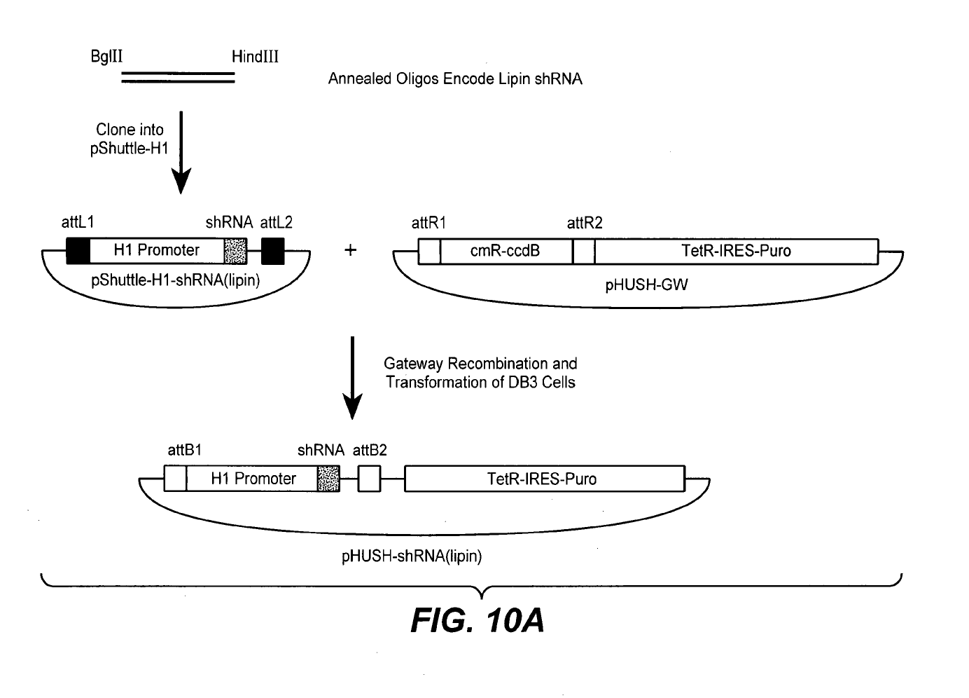

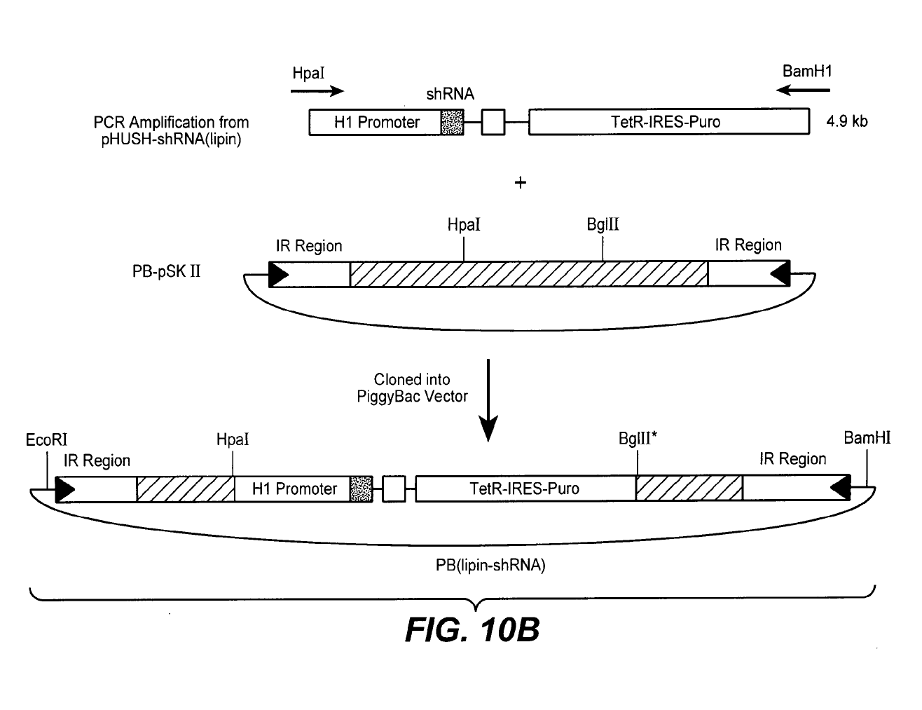

The three resulting constructs are referred to generically as pShuttle-H1-shRNA(lipin), as shown in FIG. 10A. (The H1-TetO2-2x promoter is referred to as “H1 promoter” in FIG. 10A.) The H1 promoter-shRNA cassette from pShuttle-H1-shRNA(lipin) was then subcloned into the pHUSH-GW plasmid (see U.S. application Ser. No. 11/460,606, filed Jul. 27, 2006) using Gateway recombination. As shown in FIG. 10A, the pHUSH-GW plasmid contains an attR1-cmR-ccdB-attR2 cassette upstream of a TetR-IRES-Puromycin-polyA cassette. (The TetR-IRES-Puromycin-polyA cassette is referred to as “TetR-IRES-Puro” in FIGS. 10A and 10B.) The resulting construct, which comprises the H1 promoter-shRNA cassette upstream of the TetR-IRES-Puro cassette, is referred to as “pHUSH-shRNA(lipin),” as shown in FIG. 10A. The H1 promoter-shRNA-TetR-IRES-Puro fragment was then amplified by PCR from pHUSH-shRNA(lipin) using primers having HpaI and BamHI restriction sites, as shown in FIG. 10B. The amplicon was then subcloned into HpaI and BglII sites of the piggyBac transposon in a pBluescriptSKII backbone (referred to as “PB-pSK II” in FIG. 10B) to create PB(lipin-shRNA). PiggyBac IRs corresponding to SEQ ID NOS:3 and 5 (see “Configuration 2” in Section II.A.1.b) are within the indicated regions. Transgenic mice were generated using PB(lipin-shRNA) in the same manner as described above for PB(luc-shRNA).

One skilled in the art would understand that the above-described vector can be adapted for the expression of an shRNA specific to any target nucleic acid.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference. The headings used herein are for organizational convenience and are not to be construed as limiting.

1. A nucleic acid construct comprising:
(a) a first transcription unit comprising a polynucleotide operably linked to an inducible promoter, wherein the inducible promoter comprises one or more TetO sequences;
(b) a second transcription unit comprising a coding sequence encoding a TetR; and
(c) a pair of inverted repeats, wherein one of the inverted repeats is 5′ of (a) and
(b), and the other of the inverted repeats is 3′ of (a) and (b).

2. The nucleic acid construct of claim 1, wherein the pair of inverted repeats are piggyBac inverted repeats.

3. The nucleic acid construct of claim 2, wherein the one or the other of the inverted repeats comprises a polynucleotide sequence selected from SEQ ID NO:1, SEQ ID NO:2, SEQ ID NO:3, SEQ ID NO:4, and SEQ ID NO:5.

4. The nucleic acid construct of claim 1, wherein the polynucleotide encodes a regulatory RNA.

5. The nucleic acid construct of claim 4, wherein the regulatory RNA is an shRNA.

6. The nucleic acid construct of claim 1 wherein the inducible promoter further comprises an H1 or U6 promoter.

7. The nucleic acid construct of claim 6, wherein the inducible promoter comprises at least two TetO sequences.

8. The nucleic acid construct of claim 7, wherein the inducible promoter comprises the nucleic acid sequence of SEQ ID NO:16.

9. The nucleic acid construct of claim 1, wherein the polynucleotide encodes a first RNA, and wherein the nucleic acid construct further comprises a third transcription unit, wherein the third transcription unit comprises a second polynucleotide operably linked to an inducible promoter, wherein the second polynucleotide encodes a second RNA, wherein the first RNA and the second RNA comprise sequences of at least 10 contiguous nucleotides that are complementary.

10. The nucleic acid construct of claim 1, further comprising a selectable marker.

11. The nucleic acid construct of claim 10, wherein the selectable marker is disposed in the second transcription unit.

12. The nucleic acid construct of claim 11, wherein an IRES is disposed between the coding sequence encoding a TetR and the selectable marker.

13. The nucleic acid construct of claim 1, wherein the coding sequence encoding a TetR is codon-optimized.

14. The nucleic acid construct of claim 13, wherein the coding sequence encoding a TetR comprises the nucleic acid sequence of nucleotides 1-507 of SEQ ID NO:15.

15-38. (canceled)

39. A method of expressing a polynucleotide in a transgenic mammal, the method comprising:
(a) introducing into a mammalian, non-human embryonic cell a nucleic acid construct comprising:
(i) a first transcription unit comprising the polynucleotide operably linked to an inducible promoter, wherein the polynucleotide encodes a regulatory RNA specific for an endogenous gene; and
(ii) a pair of piggyBac inverted repeats, wherein one of the inverted repeats is 5′ of (i), and the other of the inverted repeats is 3′ of (i);
(b) introducing into the mammalian, non-human embryonic cell a coding sequence encoding a piggyBac transposase that acts on the inverted repeats to mediate nucleic acid transposition;
(c) generating a transgenic mammal from the mammalian, non-human embryonic cell into which the nucleic acid construct and the coding sequence encoding the transposase have been introduced; and
(d) administering to the transgenic mammal an inducing agent that induces expression of the polynucleotide from the inducible promoter.

40-47. (canceled)

48. A transgenic non-human mammal comprising a nucleic acid construct comprising:
(a) a first transcription unit comprising a polynucleotide operably linked to an inducible promoter, wherein the polynucleotide encodes a regulatory RNA specific for an endogenous gene; and
(b) a pair of piggyBac inverted repeats, wherein one of the inverted repeats is 5′ of (a), and the other of the inverted repeats is 3′ of (a),
wherein the regulatory RNA inhibits expression of the endogenous gene in the transgenic non-human mammal.

49. The transgenic non-human mammal of claim 48, wherein the regulatory RNA is an shRNA.

50. The transgenic non-human mammal of claim 48, wherein the inducible promoter comprises one or more TetO sequences, and wherein the nucleic acid construct further comprises a second transcription unit comprising a coding sequence encoding a TetR.

51. (canceled)

52. The transgenic non-human mammal of claim 48, wherein the one or the other of the inverted repeats comprises a polynucleotide sequence selected from SEQ ID NO:1, SEQ ID NO:2, SEQ ID NO:3, SEQ ID NO:4, and SEQ ID NO:5.

53. The transgenic non-human mammal of claim 49, wherein the shRNA is specific for an endogenous gene selected from (a) a gene encoding lipin, (b) a gene encoding VEGF, or (c) a gene that is an oncogene.

54. The transgenic non-human mammal of claim 50, wherein the second transcription unit further comprises a selectable marker.

55. The transgenic non-human mammal of claim 54, wherein an IRES is disposed between the coding sequence encoding a TetR and the selectable marker.

56. The transgenic non-human mammal of claim 50, wherein the coding sequence encoding a TetR is codon-optimized.

57. The transgenic non-human mammal of claim 56, wherein the coding sequence encoding a TetR comprises the nucleic acid sequence of nucleotides 1-507 of SEQ ID NO:15.

58. The transgenic non-human mammal of claim 48, wherein the inducible promoter comprises an H1 or U6 promoter.

59. The transgenic non-human mammal of claim 50, wherein the inducible promoter comprises at least two TetO sequences.

60. The transgenic non-human mammal of claim 59, wherein the inducible promoter comprises the nucleic acid sequence of SEQ ID NO:16.

61. A cell comprising the nucleic acid construct of claim 1.

62. The cell of claim 61, wherein the cell is a mammalian cell.

63. The cell of claim 62, wherein the mammalian cell is an embryonic cell.

64. The cell of claim 62, wherein the mammalian cell is a murine cell.


SEQUENCE LISTING (28 sequences)

SEQ ID NO: 1 - DNA, 13 bp, Artificial sequence
  inverted repeat
ccctagaaagata

SEQ ID NO: 2 - DNA, 35 bp, Artificial sequence
  inverted repeat
ccctagaaagatagtctgcgtaaaattgacgcatg

SEQ ID NO: 3 - DNA, 63 bp, Artificial sequence
  inverted repeat
ccctagaaagataatcatattgtgacgtacgttaaagataatcatgcgtaaaattgacgcatg

SEQ ID NO: 4 - DNA, 63 bp, Artificial sequence
  inverted repeat
catgcgtcaattttacgcatgattatctttaacgtacgtcacaatatgattatctttctaggg

SEQ ID NO: 5 - DNA, 35 bp, Artificial sequence
  inverted repeat
catgcgtcaattttacgcagactatctttctaggg

SEQ ID NO: 6 - DNA, 29 bp, Artificial sequence
  inverted repeat
gttcaagtcggaagtttacatacacttag

SEQ ID NO: 7 - DNA, 30 bp, Artificial sequence
  inverted repeat
cagtgggtcagaagtttacatacactaagg

SEQ ID NO: 8 - DNA, 31 bp, Artificial sequence
  inverted repeat
cagtgggtcagaagttaacatacactcaatt

SEQ ID NO: 9 - DNA, 30 bp, Artificial sequence
  inverted repeat
agttgaatcggaagtttacatacaccttag

SEQ ID NO: 10 - DNA, 226 bp, Artificial sequence
  inverted repeat
agttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 11 - DNA, 228 bp, Artificial sequence
  inverted repeat
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaatgtatttggctaaggtgtatgtaaacttccgacttcaactg

SEQ ID NO: 12 - DNA, 21 bp, Artificial sequence
  inverted repeat
tgtgaaaaagtgtttgccccc

SEQ ID NO: 13 - DNA, 250 bp, Artificial sequence
  inverted repeat
cagtggtgtgaaaaagtgtttgcccccttcctcatttcctgttcctttgcatgtttgtcacacttaagtgtttcggaacatcaaaccaatttaaacaatagtcaaggacaacacaagtaaacacaaaatgcaatttgtaaatgaaggtgtttattattaaaggtgaaaaaaaatccaaaccatcatggccctgtgtgaaaaagtgattgccccccttgttaaaacatactataactgtggttgtccacac

SEQ ID NO: 14 - protein, 594 aa, Trichopulsia ni
MGSSLDDEHILSALLQSDDELVGEDSDSEISDHVSEDDVQSDTEEAFIDEVHEVQPTSSGSEILDEQNVIEQPGSSLASNKILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVRGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSREKFMRNLYMSLTSSFMRKRLEAPTLKRYLRDNISNILPNEVPGTSDDSTEEPVTKKRTYCTYCPSKIRRKANASCKKCKKVICREHNIDMCQSCF

SEQ ID NO: 15 - DNA, 656 bp, Artificial sequence
  codon-optimized TetR
atgtccagactggataagtccaaggtgattaattccgctctggaactcctgaacgaggtcggcatcgagggactgaccacacggaagctggctcagaaactcggcgtcgaacagcctaccctctactggcatgtcaaaaataagagagccctcctggacgccctggctatcgagatgctggacagacaccacacccacttctgccccctggaaggcgaatcctggcaggatttcctccggaacaacgctaaaagctttagatgcgccctcctcagccatagagacggagctaaagtgcacctgggaacccggcctacagaaaaacagtacgagacactggaaaaccagctcgctttcctctgccaacaaggctttagcctggaaaacgccctctacgctctcagcgctgtcggccattttacactgggctgcgtgctcgaggaccaggagcaccaagtggctaaagagagcgggaaacccctaccaccgatagcatgccccccctgctgagacaagccattgagctctttgatcatcagggagctgaacccgccttcctctttggactcgaactcattatttgcggactcgagaagcaactgaaatgcgaaagcggaagcgcctactccggctccagagaatttcggtcctactag

SEQ ID NO: 16 - DNA, 225 bp, Artificial sequence
  H1-tetO2-2X promoter
cgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagatcccc

SEQ ID NO: 17 - DNA, 9611 bp, Artificial sequence
  PB(luc-shRNA) construct
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattcaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaactatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcgttactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttgaaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttaaccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagatcccccgatatgggctgaatacaattcaagagattgtattcagcccatatcgttttttggaaaagcttatcgataccgtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccactagtaaagggaaaaccggtgttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccatgtccagactggataagtccaaggtgattaattccgctctggaactcctgaacgaggtcggcatcgagggactgaccacacggaagctggctcagaaactcggcgtcgaacagcctaccctctactggcatgtcaaaaataagagagccctcctggacgccctggctatcgagatgctggacagacaccacacccacttctgccccctggaaggcgaatcctggcaggatttcctccggaacaacgctaaaagctttagatgcgccctcctcagccatagagacggagctaaagtgcacctgggaacccggcctacagaaaaacagtacgagacactggaaaaccagctcgctttcctctgccaacaaggctttagcctggaaaacgccctctacgctctcagcgctgtcggccattttacactgggctgcgtgctcgaggaccaggagcaccaagtggctaaagaggagcgggaaacccctaccaccgatagcatgccccccctgctgagacaagccattgagctctttgatcatcagggagctgaacccgccttcctctttggactcgaactcattatttgcggactcgagaagcaactgaaatgcgaaagcggaagcgcctactccggctccagagaatttcggtcctactaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggtgcattctagttgtgggatctgacaatgttcagtgcagagactcggctacgcctcgtggactttgaagttgaccaacaatgtttattcttacctctaatagtcctctgtggcaaggtcaagattttgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaacgcacagcgacggattcgcgctatttagaaagagagagcaatatttcaagaatgcatgcgtcaattttacgcagactatctttctagggttaaaaaagatttgcgctggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 18 - DNA, 4965 bp, Artificial sequence
  pCAG-PBase
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacagtagcttccaccatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacaaaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacaaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaacgaaaaaacgtacttactgtacttactgcccctctaaaataaggcgaaaggcaaatgcatcgtgcaaaaaatgcaaaaaagttatttgtcgagagcataatattgatatgtgccaaagttgtttctgagaattcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaaatttttcaaaagtagttgacaattaatcatcggcatagtatatcggcatagtataatacgactcactataggagggccatcatggccaagttgaccagtgctgtcccagtgctcacagccagggatgtggctggagctgttgagttctggactgacaggttggggttctccagagattttgtggaggatgactttgcaggtgtggtcagagatgatgtcaccctgttcatctcagcagtccaggaccaggtggtgcctgacaacaccctggcttgggtgtgggtgagaggactggatgagctgtatgctgagtggagtgaggtggtctccaccaacttcagggatgccagtggccctgccatgacagagattggagagcagccctgggggagagagtttgccctgagagacccagcaggcaactgtgtgcactttgtggcagaggagcaggactgaggataagaattgagtttcagaaaagggggcctgagtggccccttttttcaacttaattaannnnnnnnnn

SEQ ID NO: 19 - DNA, 68 bp, Artificial sequence
  Oligonucleotide
acgtgaaatccctatcagtgatagagacttataagttccctatcagtgatagagatctaaagggaaaa

SEQ ID NO: 20 - DNA, 72 bp, Artificial sequence
  Oligonucleotide
agctttttccctttagatctctatcactgatagggaacttataagtctctatcactgatagggatttcacgt

SEQ ID NO: 21 - protein, 218 aa, Artificial sequence
  codon-optimized tetR
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSGSREFRSY

SEQ ID NO: 22 - protein, 212 aa, Artificial sequence
  Puromycin selectable marker
MISLPQPTRRRPSMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 23 - DNA, 65 bp, Artificial sequence
  Oligonucleotide
gatcccccgacaaccctgctatcatcttcaagagagatgatagcagggttgtcgttttttggaaa

SEQ ID NO: 24 - DNA, 65 bp, Artificial sequence
  Oligonucleotide
agcttttccaaaaaacgacaaccctgctatcatctctcttgaagatgatagcagggttgtcgggg

SEQ ID NO: 25 - DNA, 65 bp, Artificial sequence
  Oligonucleotide
gatccccggttgacgccaaagaataattcaagagattattctttggcgtcaaccttttttggaaa

SEQ ID NO: 26 - DNA, 65 bp, Artificial sequence
  Oligonucleotide
agcttttccaaaaaaggttgacgccaaagaataatctcttgaattattctttggcgtcaaccggg

SEQ ID NO: 27 - DNA, 65 bp, Artificial sequence
  Oligonucleotide
gatccccccggaagactcctgataaattcaagagatttatcaggagtcttccggttttttggaaa

SEQ ID NO: 28 - DNA, 65 bp, Artificial sequence
  Oligonucleotide
agcttttccaaaaaaccggaagactcctgataaatctcttgaatttatcaggagtcttccggggg